SEQUENCE LISTING (32 sequences)

SEQ ID NO: 1 - RNA, 14 nt, ARTIFICIAL SEQUENCE
  synthetic oligonucleotide
uunuunuunuunuu

SEQ ID NO: 2 - RNA, 21 nt, ARTIFICIAL SEQUENCE
  synthetic oligonucleotide
uuuuuuuuuuuuuuuuuuuuu

SEQ ID NO: 3 - RNA, 15 nt, ARTIFICIAL SEQUENCE
  synthetic oligonucleotide
uuuuuuuuuuuuuuu

SEQ ID NO: 4 - RNA, 10 nt, ARTIFICIAL SEQUENCE
  synthetic oligonucleotide
uuuuuuuuuu

SEQ ID NO: 5 - RNA, 21 nt, ARTIFICIAL SEQUENCE
  synthetic oligonucleotide
uuuuuuuuuuuuuuuuuuuuu

SEQ ID NO: 6 - RNA, 15 nt, ARTIFICIAL SEQUENCE
  synthetic oligonucleotide
uuuuuuuuuuuuuuu

SEQ ID NO: 7 - RNA, 10 nt, ARTIFICIAL SEQUENCE
  synthetic oligonucleotide
uuuuuuuuuu

SEQ ID NO: 8 - RNA, 21 nt, ARTIFICIAL SEQUENCE
  synthetic oligonucleotide
uuuuuuuuuuuuuuuuuuuuu

SEQ ID NO: 9 - RNA, 21 nt, ARTIFICIAL SEQUENCE
  synthetic oligonucleotide
uuuuuuuuuuuuuuuuuuuuu

SEQ ID NO: 10 - RNA, 21 nt, ARTIFICIAL SEQUENCE
  synthetic oligonucleotide
uuuuuuuuuuuuuuuuuuuuu

SEQ ID NO: 11 - RNA, 20 nt, ARTIFICIAL SEQUENCE
  synthetic oligonucleotide
gcccgucuguugugugacuc

SEQ ID NO: 12 - RNA, 21 nt, ARTIFICIAL SEQUENCE
  synthetic oligonucleotide
ccuuuccuuucuuuccuuucc

SEQ ID NO: 13 - RNA, 21 nt, ARTIFICIAL SEQUENCE
  synthetic oligonucleotide
cccccccuuucuuuccuuucc

SEQ ID NO: 14 - RNA, 21 nt, ARTIFICIAL SEQUENCE
  synthetic oligonucleotide
cccccccuuucuuuccccccc

SEQ ID NO: 15 - RNA, 21 nt, ARTIFICIAL SEQUENCE
  synthetic oligonucleotide
uuucccuuucccuuucccuuu

SEQ ID NO: 16 - RNA, 21 nt, ARTIFICIAL SEQUENCE
  synthetic oligonucleotide
ccuuccuuccuuccuuccuuc

SEQ ID NO: 17 - RNA, 21 nt, ARTIFICIAL SEQUENCE
  synthetic oligonucleotide
uucccuucccuuccuucccuu

SEQ ID NO: 18 - RNA, 21 nt, ARTIFICIAL SEQUENCE
  synthetic oligonucleotide
cccccuuucuuucuuuccccc

SEQ ID NO: 19 - RNA, 21 nt, ARTIFICIAL SEQUENCE
  synthetic oligonucleotide
ccccuuuccuuuccuuucccc

SEQ ID NO: 20 - RNA, 21 nt, ARTIFICIAL SEQUENCE
  synthetic oligonucleotide
cccuuucccuuucccuuuccc

SEQ ID NO: 21 - RNA, 21 nt, ARTIFICIAL SEQUENCE
  synthetic oligonucleotide
ccuuuccccuuuccccuuucc

SEQ ID NO: 22 - RNA, 21 nt, ARTIFICIAL SEQUENCE
  synthetic oligonucleotide
cuuucccccuuucccccuuuc

SEQ ID NO: 23 - RNA, 21 nt, ARTIFICIAL SEQUENCE
  synthetic oligonucleotide
cucucucucucucucucucuc

SEQ ID NO: 24 - RNA, 21 nt, ARTIFICIAL SEQUENCE
  synthetic oligonucleotide
ccccuucuucuucuucuuccc

SEQ ID NO: 25 - RNA, 21 nt, ARTIFICIAL SEQUENCE
  synthetic oligonucleotide
ccccccuuuuuuuuuuccccc

SEQ ID NO: 26 - RNA, 21 nt, ARTIFICIAL SEQUENCE
  synthetic oligonucleotide
aaaaaaaaaaaaaaaaaaaaa

SEQ ID NO: 27 - RNA, 21 nt, ARTIFICIAL SEQUENCE
  synthetic oligonucleotide
ccccccccccccccccccccc

SEQ ID NO: 28 - DNA, 21 bp, ARTIFICIAL SEQUENCE
  synthetic oligonucleotide
ttttttttttttttttttttt

SEQ ID NO: 29 - RNA, 21 nt, ARTIFICIAL SEQUENCE
  synthetic oligonucleotide
uuunnnuuunnnuuunnnuuu

SEQ ID NO: 30 - RNA, 21 nt, ARTIFICIAL SEQUENCE
  synthetic oligonucleotide
nnncccnnncccnnncccnnn

SEQ ID NO: 31 - DNA, 5007 bp, Homo sapiens
actccagatataggatcactccatgccatcaagaaagttgatgctattgggcccatctcaagctgatcttggcacctctcatgctctgctctcttcaaccagacctctacattccattttggaagaagactaaaaatggtgtttccaatgtggacactgaagagacaaattcttatcctttttaacataatcctaatttccaaactccttggggctagatggtttcctaaaactctgccctgtgatgtcactctggatgttccaaagaaccatgtgatcgtggactgcacagacaagcatttgacagaaattcctggaggtattcccacgaacaccacgaacctcaccctcaccattaaccacataccagacatctccccagcgtcctttcacagactggaccatctggtagagatcgatttcagatgcaactgtgtacctattccactggggtcaaaaaacaacatgtgcatcaagaggctgcagattaaacccagaagctttagtggactcacttatttaaaatccctttacctggatggaaaccagctactagagataccgcagggcctcccgcctagcttacagcttctcagccttgaggccaacaacatcttttccatcagaaaagagaatctaacagaactggccaacatagaaatactctacctgggccaaaactgttattatcgaaatccttgttatgtttcatattcaatagagaaagatgccttcctaaacttgacaaagttaaaagtgctctccctgaaagataacaatgtcacagccgtccctactgttttgccatctactttaacagaactatatctctacaacaacatgattgcaaaaatccaagaagatgattttaataacctcaaccaattacaaattcttgacctaagtggaaattgccctcgttgttataatgccccatttccttgtgcgccgtgtaaaaataattctcccctacagatccctgtaaatgcttttgatgcgctgacagaattaaaagttttacgtctacacagtaactctcttcagcatgtgcccccaagatggtttaagaacatcaacaaactccaggaactggatctgtcccaaaacttcttggccaaagaaattggggatgctaaatttctgcattttctccccagcctcatccaattggatctgtctttcaattttgaacttcaggtctatcgtgcatctatgaatctatcacaagcattttcttcactgaaaagcctgaaaattctgcggatcagaggatatgtctttaaagagttgaaaagctttaacctctcgccattacataatcttcaaaatcttgaagttcttgatcttggcactaactttataaaaattgctaacctcagcatgtttaaacaatttaaaagactgaaagtcatagatctttcagtgaataaaatatcaccttcaggagattcaagtgaagttggcttctgctcaaatgccagaacttctgtagaaagttatgaaccccaggtcctggaacaattacattatttcagatatgataagtatgcaaggagttgcagattcaaaaacaaagaggcttctttcatgtctgttaatgaaagctgctacaagtatgggcagaccttggatctaagtaaaaatagtatattttttgtcaagtcctctgattttcagcatctttctttcctcaaatgcctgaatctgtcaggaaatctcattagccaaactcttaatggcagtgaattccaacctttagcagagctgagatatttggacttctccaacaaccggcttgatttactccattcaacagcatttgaagagcttcacaaactggaagttctggatataagcagtaatagccattattttcaatcagaaggaattactcatatgctaaactttaccaagaacctaaaggttctgcagaaactgatgatgaacgacaatgacatctcttcctccaccagcaggaccatggagagtgagtctcttagaactctggaattcagaggaaatcacttagatgttttatggagagaaggtgataacagatacttacaattattcaagaatctgctaaaattagaggaattagacatctctaaaaattccctaagtttcttgccttctggagtttttgatggtatgcctccaaatctaaagaatctctctttggccaaaaatgggctcaaatctttcagttggaagaaactccagtgtctaaagaacctggaaactttggacctcagccacaaccaactgaccactgtccctgagagattatccaactgttccagaagcctcaagaatctgattcttaagaataatcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacctgtgatgctgtgtggtttgtctggtgggttaaccatacggaggtgactattccttacctggccacagatgtgacttgtgtggggccaggagcacacaagggccaaagtgtgatctccctggatctgtacacctgtgagttagatctgactaacctgattctgttctcactttccatatctgtatctctctttctcatggtgatgatgacagcaagtcacctctatttctgggatgtgtggtatatttaccatttctgtaaggccaagataaaggggtatcagcgtctaatatcaccagactgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccctttcagaagtccaagttcctccagctccggaaaaggctctgtgggagttctgtccttgagtggccaacaaacccgcaagctcacccatacttctggcagtgtctaaagaacgccctggccacagacaatcatgtggcctatagtcaggtgttcaaggaaacggtctagcccttctttgcaaaacacaactgcctagtttaccaaggagaggcctggctgtttaaattgttttcatatatatcacaccaaaagcgtgttttgaaattcttcaagaaatgagattgcccatatttcaggggagccaccaacgtctgtcacaggagttggaaagatggggtttatataatgcatcaagtcttctttcttatctctctgtgtctctatttgcacttgagtctctcacctcagctcctgtaaaagagtggcaagtaaaaaacatggggctctgattctcctgtaattgtgataattaaatatacacacaatcatgacattgagaagaactgcatttctacccttaaaaagtactggtatatacagaaatagggttaaaaaaaactcaagctctctctatatgagaccaaaatgtactagagttagtttagtgaaataaaaaaccagtcagctggccgggcatggtggctcatgcttgtaatcccagcactttgggaggccgaggcaggtggatcacgaggtcaggagtttgagaccagtctggccaacatggtgaaaccccgtctgtactaaaaatacaaaaattagctgggcgtggtggtgggtgcctgtaatcccagctacttgggaggctgaggcaggagaatcgcttgaacccgggaggtggaggtggcagtgagccgagatcacgccactgcaatgcagcccgggcaacagagctagactgtctcaaaagaacaaaaaaaaaaaaacacaaaaaaactcagtcagcttcttaaccaattgcttccgtgtcatccagggccccattctgtgcagattgagtgtgggcaccacacaggtggttgctgcttcagtgcttcctgctctttttccttgggcctgcttctgggttccatagggaaacagtaagaaagaaagacacatccttaccataaatgcatatggtccacctacaaatagaaaaatatttaaatgatctgcctttatacaaagtgatattctctacctttgataatttacctgcttaaatgtttttatctgcactgcaaagtactgtatccaaagtaaaatttcctcatccaatatctttcaaactgttttgttaactaatgccatatatttgtaagtatctgcacacttgatacagcaacgttagatggttttgatggtaaaccctaaaggaggactccaagagtgtgtatttatttatagttttatcagagatgacaattatttgaatgccaattatatggattcctttcattttttgctggaggatgggagaagaaaccaaagtttatagaccttcacattgagaaagcttcagttttgaacttcagctatcagattcaaaaacaacagaaagaaccaagacattcttaagatgcctgtactttcagctgggtataaattcatgagttcaaagattgaaacctgaccaatttgctttatttcatggaagaagtgatctacaaaggtgtttgtgccatttggaaaacagcgtgcatgtgttcaagccttagattggcgatgtcgtattttcctcacgtgtggcaatgccaaaggctttactttacctgtgagtacacactatatgaattatttccaacgtacatttaatcaataagggtcacaaattcccaaatcaatctctggaataaatagagaggtaattaaattgctggagccaactatttcacaacttctgtaagc

SEQ ID NO: 32 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

(19) United States
(12) Patent Application Publication
(10) Pub. No.: US 2009/0169472 A1
(43) Pub. Date: Jul. 2, 2009

(54) METHODS AND COMPOSITIONS FOR TREATING IMMUNE DISORDERS

(75) Inventors: Sandra Diebold, London (GB); Caetano Reis E Sousa, London (GB); Carine Paturel, Dardilly (FR)

Correspondence Address: SALIWANCHIK LLOYD & SALIWANCHIK;A PROFESSIONAL ASSOCIATION, PO Box 142950, GAINESVILLE, FL, 32614, US

(73) Assignee: CANCER RESEARCH TECHNOLOGY LTD., London (GB)

(21) Appl. No.: 12/088,752

(22) PCT Filed: Oct. 12, 2006
(86) PCT No.: PCT/EP06/67334

§ 371 Date: Mar. 31, 2008

Related U.S. Application Data
(60) Provisional application No. 60/726,305, filed on Oct. 12, 2005.
(60) Provisional application No. 60/751,917, filed on Dec. 20, 2005.

(51) Int. Cl. A61K 51/00; C07H 21/00; A61K 31/7088; A61K 9/127; A61K 39/395; A61K 39/00; C12N 5/02; A61F 13/00; C12Q 1/68

(52) U.S. Cl. 424/1.65; 536/23.1; 514/44; 424/450; 424/141.1; 424/184.1; 435/375; 424/422; 435/6

(57) ABSTRACT

The present invention provides oligonucleotides, compositions comprising them and methods that use the oligonucleotides and compositions for stimulating cells expressing the TLR7 and/or TLR8 receptor. The oligonucleotides comprise for stimulating TLR7 comprise uracil-rich regions. The oligonucleotides for stimulating TLR8 comprise guanine-rich regions. The present methods and compositions are useful, inter alia, for treating or preventing conditions such as infectious disease and cancer.

### FIELD OF THE INVENTION

The present invention generally relates to the field of immunology. More particularly, the invention relates to compositions and methods for altering immune function, particularly by stimulating receptors such as the Toll-like receptor 7 (TLR7) and Toll-like receptor 8 (TLR8) present in the membranes of cells such as plasmacytoid dendritic cells.

### BACKGROUND

One of the earliest responses to influenza and other viruses is the production of type I IFNs, critical cytokines that establish an antiviral state and bridge the innate and adaptive immune systems (Le Bon et al., (2002) Curr. Opin. Immunol. 14:432). The mammalian innate immune system recognizes the presence of invading pathogens by a family of receptors belonging to the Toll-like receptor (TLR) family. TLRs such as TLR3, TLR7, TLR8 and TLR9—all involved in recognizing viral pathogen-associated molecular patterns (PAMPs)—are expressed intracellularly and sample the content of endosomes for the presence of viral PAMPs among extracellular material that these cells have taken up. In case of CD8a+ dendritic cells (DCs) that take up material from apoptotic cells, these TLRs sense viral PAMPs present in infected cells, while plasmacytoid DC seem to take up virus particles rather than cellular material and recognize the genomic nucleic acids inside the virus particles upon uptake.

Several characteristics of the viral genome, such as double-stranded RNA (dsRNA) and high CpG content, can serve as molecular signatures that can be distinguished by the host as nonself. The host-virus interactions that lead to the secretion of type I IFNs by the infected cells, likely involving pattern recognition through TLRs. Although most types of cells can produce IFNα and IFNβ on viral infection, plasmacytoid dendritic cells (pDCs) are particularly adept at secreting very high levels of type I IFNs in response to certain viruses.

As members of the pro-inflammatory interleukin-1 receptor (IL-1R) family, TLRs share homologies in their cytoplasmic domains called Toll/L-1R homology (TIR) domains (see e.g., PCT published applications PCT/US98/08979 and PCT/US01/16766; the entire disclosures of which are herein incorporated by reference). Intracellular signaling mechanisms mediated by TLRs appear generally similar, with MyD88 and tumor necrosis factor receptor-associated factor 6 (TRAF6) believed to have critical roles (Wesche H et al. (1997) Immunity 7:837-47; Medzhitov R et al. (1998) Mol Cell 2:253-8; Adachi 1 et al. (1998) Immunity 9:143-50; Kawai Tetal. (1999) Immunity 11:115-22); Cao Z et al. (1996) Nature 383:443-6; Lomaga M A et al. (1999) Genes Dev 13:1015-24; the entire disclosures of which are herein incorporated by reference). Signal transduction between MyD88 and TRAF6 is known to involve members of the serine-threonine kinase IL-1 receptor-associated kinase (IRAK) family, including at least IRAK-1 and IRAK-2 (Muzio M et al. (1997) Science 278:1612-5).

Upon activation of TLRs, the Toll homology domain of MyD88 binds the TIR domain of the TLR, and the death domain of MyD88 binds the death domain of the serine kinase IRAK. IRAK interacts with TRAF6, which acts as an entryway into at least two pathways, one leading to activation of the transcription factor NF-kB, and the other leading to activation of Jun and Fos, members of the activator protein-1 (AP-1) transcription factor family. Activation of NF-kB involves the activation of TAK-1, a member of the MAP 3 kinase (MAPK) family, and IkB kinases. The IkB kinases phosphorylate IkB, leading to its degradation and the translocation of NF-kB to the nucleus. Activation of Jun and Fos is believed to involve MAP kinase kinases (MAPKKs) and MAP kinases ERK, p38, and JNK/SAPK. Both NF-kB and AP-1 are involved in controlling the transcription of a number of key immune response genes, including genes for various cytokines and costimulatory molecules (see, e.g., Aderem A et al. (2000) Nature 406:782-7; Haicker H et al. (1999) EMBO J. 18:6973-82).

Ligands for many but not all of the TLRs have been described. For instance, it has been reported that TLR2 signals in response to peptidoglycan and lipopeptides (Yoshimura A et al. (1999) J Immunol 163:1-5; Brightbill H D et al. (1999) Science 285:732-6; Aliprantis A O et al. (1999) Science 285:736-9; Takeuchi et al. (1999) Immunity 11:443-51; Underhill D M et al. (1999) Nature 401:811-5). TLR4 has been reported to signal in response to lipopolysaccharide (LPS) (Hoshino K et al. (1999) J Immunol 162:3749-52; Poltorak A et al. (1998) Science 282:2085-8; Medzhitov R et al. (1997) Nature 388:394-7). Bacterial flagellin has been reported to be a natural ligand for TLR5 (Hayashi F et al. (2001) Nature 410:1099-1103). TLR6, in conjunction with TLR2, has been reported to signal in response to proteoglycans (Ozinsky et al. (2000) PNAS 97:13766-71; Takeuchi et al. (2001) Int Immunol 13:933-40).

TLR7 is a pattern recognition receptor for detection of genomic viral RNA. TLR7-mediated IFNα induction in plasmacytoid dendritic cells (PDC) can be triggered by viral RNA, mammalian mRNA and in vitro transcribed GFP RNA irrespective of the RNA sequence. A variety of sequences have previously been shown to be capable of stimulating TLR7 on PDCs to some degree, including long strands of polyU of variable length (Diebold et al. (2004) Science 303: 1529), oligonucleotides containing a high proportion of GU nucleotides (Heil et al. (2004) Science 303: 1526; U.S. Patent application US2003/0232074), certain specific siRNA sequences (Hornung et al. (2005) Nature Med. 11:263); and guanine nucleotide analogs (Lee et al (2003) PNAS 100: 6646-6651). It has been reported that certain antiviral imidazoquinoline compounds, such as imiquimod and resiquimod (R848), can activate TLR7 (Hemmi H et al. (2002) Nat Immunol 3:196-200; Jurk M et al. (2002) Nat Immunol 3:499).

TLR8 is a pattern recognition receptor for detection of single-stranded RNA. It appears to be functional in human dendritic cells, particularly myeloid dendritic cells, but not in mouse dendritic cells (Jurk M et al. (2002) Nat Immunol 3:499). TLR8 also is functional in CD4⁺ regulatory T-cells. GU-rich ribonucleotides and deoxyribonucleotides, guanine nucleotide analogs and imidazoquinoline compounds, such as imiquimod and resiquimod (R848) which stimulate TLR7 have also been shown to stimulate human TLR8. The significance of the lack of TLR8 in mice and why TLR7 and TLR8 appear to possess somewhat redundant recognition functions in human immune cells is not known.

In view of the importance of TLR7- and TLR8-mediated stimulation of the innate immune response for the defense against viruses and other infectious agents, and generally for stimulating the immune response to help treat and prevent conditions such as cancer, there is a great need in the art for novel compounds capable of effectively and reliably activating TLR7 and TLR8 independently of one another in vitro and in vivo. The present invention addresses this and other needs.

### SUMMARY OF THE INVENTION

In one aspect, the present invention provides isolated single-stranded oligonucleotides, compositions which comprise them and methods for optimally stimulating TLR-mediated signaling, specifically through the TLR7 receptor. The oligonucleotides, compositions and methods described herein are useful for enhancing the activation of TLR7-expressing cells, e.g. dendritic cells such as plasmacytoid dendritic cells, and certain subsets of regulatory T-cells, in vitro and in vivo. Such oligonucleotides, compositions and methods are useful in a number of clinical applications, including as pharmaceutical agents and methods for treating or preventing conditions such as cancer or infectious diseases, particularly viral infections. The oligonucleotides and compositions of the invention can also be used in methods for assessing the effects of other compounds on TLR7 activity, e.g., in assays to identify or characterize other candidate modulators of TLR7 or of TLR7-expressing cells. The oligonucleotides and compositions are also useful in methods of inducing IFNα production and/or release, particularly by dendritic cells.

The presently described oligonucleotides are based on studies presented herein in which various structural parameters were varied in order to determine those most important for TLR7 stimulation. Surprisingly, it was discovered that the nucleotide uridine is the essential feature determining recognition by and activation of TLR7 receptors. Accordingly, in one embodiment, the present invention provides a single stranded oligonucleotide consisting of between 10 and 50 nucleotides and comprising a sequence selected from: UUUᵣ-(X)ₙ-UUUᵣ, or UUᵣ-X-UUᵣ-X-UUᵣ, wherein each U is an independently selected uracil-containing nucleotide; each X is independently selected from any nucleotide, optionally a non-uracil nucleotide or a uracil; r is an integer from 1 to 20, preferably from 1 to 10 and preferably 1, 2, 3, 4 or 5, and n is an integer from 1 to 4, wherein said oligonucleotide comprises at least one non-uracil-containing nucleotide or at least one non-natural linkage.

In a preferred embodiment, the nucleotides (e.g. non-uracil nucleotides, derivatives) of the invention do not confer upon the oligonucleotide the ability to induce substantial amounts of IL-6 when brought into contact with a biological sample, preferably a sample comprising a dendritic cell (e.g. where pDC or other TLR7 expressing DC are present). Preferably, TLR7 agonists according to the invention are selected for their ability to induce IFN-alpha as opposed to IL-6, and oligonucleotides with the greatest ratio of IFN-alpha:IL-6 induction are preferred, particularly for the treatment of e.g. infectious disease.

For example, it was discovered that short oligonucleotides of defined length consisting entirely of uridine or deoxyuridine nucleotides possess potent TLR7-stimulating ability. Thus, in one preferred embodiment each of the nucleotides in said oligonucleotide is a uracil-containing oligonucleotide and said oligonucleotide comprises at least one non-natural backbone bond. More preferably, each nucleotide is uridine. It was also discovered that oligonucleotides containing two or more triplets of uridines, or five or more doublets of uridines, are also potent activators, particularly when the doublets or triplets are separated by a small number, preferably one, of intervening nucleotides. Accordingly, in another preferred embodiment, said oligonucleotide comprises the sequence (UUUᵣ-(X)ₙ)ₘ or (UUUU-(X)ₙ)ₘ, wherein X is any nucleotide, m is an integer greater than two. Optionally X is a non-uracil nucleotide; optionally X is a uridine, and r is an integer from 1 to 20, preferably from 1 to 10 and preferably 1, 2, 3, 4 or 5. Preferably, m is 3 or 4. More preferably, each U is of uridine. Even more preferably, each n is 1. It was also found that stretches of more than five, preferably ten, consecutive uridines within an oligonucleotide is sufficient to confer strong TLR7-activating ability. Thus, in another embodiment, the present invention provides a single stranded oligonucleotide consisting of between 10 and 50 nucleotides and comprising the sequence: Y(U)ₚY, wherein each U is independently selected from a uracil-containing nucleotides, each Y is independently selected from a non-uracil-containing nucleotide; and p is an integer greater than 4. More preferred is when p is an integer greater than 5, 6, 7, 8, 9, 10, 11 or 12. In each of these described embodiments, it is preferred that each U is uridine.

It was also found that an oligonucleotide comprising five uridine doublets each separated by a single non-uridine nucleotide and a similarly sized oligonucleotide with ten consecution uridines were both equal in their ability to stimulate TLR7 as an oligonucleotide of the same size consisting entirely of uridines. Thus, according to another preferred embodiment, the oligonucleotide comprises the sequence:

| | |
|---|---|
| UUXUUXUUXUUXUU. | (SEQ ID NO 1) |

It was also found that these sequence features hold independent of the backbone of the oligonucleotide. For example, the oligonucleotide can be comprised of either RNA or DNA nucleotides. Also, oligonucleotides comprising phosphorothioate linkages work as effectively as those comprising phosphodiester linkages. Phosphorothioate and other non-natural linkages impart enhanced stability to oligonucleotides comprising such linkages. Thus, the presence of one or more of such non-natural linkages are preferred in any of the oligonucleotides described above. In a preferred embodiment, at least one non-natural linkage is a phosphorothioate linkage.

In one embodiment, all of the nucleotides in the oligonucleotide are ribonucleotides. In another embodiment, all of the nucleotides in the oligonucleotide are deoxyribonucleotides. In another embodiment, the length of the oligonucleotide is between 10 to 30 nucleotides. In another embodiment, the oligonucleotide is between 15 and 30 nucleotides in length. In yet another embodiment, the oligonucleotide is between 15 and 21 nucleotides in length. In yet another embodiment, the oligonucleotide is between 21 and 30 nucleotides in length. Preferably the oligonucleotide is 15, or 21 nucleotides in length. In an even more preferred embodiment, the oligonucleotide is 21 nucleotides in length. In another embodiment, a majority of uracil-containing nucleotides within the oligonucleotide are adjacent to at least one other uracil-containing nucleotide.

In another embodiment, the oligonucleotide comprises a sequence selected from the group consisting of SSD8 (SEQ ID NO 12), SSD9 (SEQ ID NO 13), SSD10 (SEQ ID NO 14), SSD21 (SEQ ID NO 18), SSD22 (SEQ ID NO 19), SSD23 (SEQ ID NO 20), SSD24 (SEQ ID NO 21), SSD28 (SEQ ID NO 24), SSD29 (SEQ ID NO 25), polyUs-21 (SEQ ID NO 5), polyUs-15 (SEQ ID NO 6) or polyUs-10 (SEQ ID NO 7). In another embodiment, the nucleotide sequence of the oligonucleotide consists of a sequence selected from the group consisting of SSD8, SSD9, SSD10, SSD21, SSD22, SSD23, SSD24, SSD28, SSD29, polyUs-21, polydUs21 (SEQ ID NO 9), polyUs-15 or polyUs-10 (SEQ ID NO 4). In yet another embodiment, the oligonucleotide comprises a nucleotide sequence selected from polyUo15, polyUo21 (SEQ ID NO 8), or polydUs21 (SEQ ID NO 9), wherein said oligonucleotide comprises at least one non-uracil-containing base or at least non-natural linkage. In yet another embodiment, the oligonucleotide additionally comprises at least one CG dinucleotide, wherein C is an unmethylated cytosine-containing nucleotide, and G is a guanine-containing nucleotide. The CG doublet may be present as part of a sequence selected from UUU-(X)ₙ-UUU, UU-X-UU-X-UU, or Y(U)ₚY, or outside of those sequences. Such a sequence is known to agonize the TLR9 receptor which will be desirable in certain therapeutic and other uses of the oligonucleotides of this invention. In an alternate embodiment, the oligonucleotide specifically excludes any CG doublets. Such oligonucleotides do not agonize the TLR9 receptor. Avoiding agonism of the TLR9 receptor will be desirable in specific therapeutic and other uses of the oligonucleotides of this invention.

In one example, the oligonucleotide of the invention is a TLR7 agonist which induces apoptosis in a target cell. The compound imiquimod, an agonist of TLR7 and TLR8, as well as TLR3 agonists have been reported to induce apoptosis (Meyer T, Nindl I, Schmook T, Ulrich C, Sterry W, Stockfleth E. Induction of apoptosis by Toll-like receptor-7 agonist in tissue cultures. Br J. Dermatol. 2003 November; 149 Suppl 66:9-14.; Schon et al. (2004) J. Invest. Dermatol. 122:1266-1276; and WO/2006054177 (Andre et al)). In one embodiment, the inventors provide that the oligonucleotide of the invention can be used to induce apoptosis of a target cell, including in one preferred embodiment, a cell expressing a TLR7 polypeptide. The cell is preferably a tumor cell. Thus, in one aspect, the invention provides determining whether a cell, preferably a tumor cell, expresses a TLR7 polypeptide, and if said tumor cell expresses the TLR7 polypeptide, bringing an oligonucleotide of the invention into contact with said cell in an amount effective to induce apoptosis of the cell. In another embodiment, the invention provides determining whether a cell, preferably a tumor cell, in an individual expresses a TLR7 polypeptide, and if said tumor cell expresses the TLR7 polypeptide, administering said oligonucleotide of the invention to said individual in an amount effective to induce apoptosis of the cell.

In yet further embodiments, an oligonucleotide comprises at least one CG dinucleotide, wherein C is an unmethylated cytosine-containing nucleotide, and G is a guanine-containing nucleotide, and said oligonucleotide does not contain any of the uridine containing sequences described herein, including, UUUU, UUU-(X)ₙ-UUU, UU-X-UU-X-UU, or Y(U)ₚY. Such an oligonucleotide will agonize the TLR9 receptor without agonizing the TLR7 receptor.

The present invention also provides a composition comprising an isolated single stranded oligonucleotide of between 10 and 50 nucleotides in length, and comprising a sequence selected from: UUU-(X)ₙ-UUU, UU-X-UU-X-UU, or Y(U)ₚY, wherein each U is independently selected from a uracil-containing nucleotide; each X is independently selected from any nucleotide; each Y is independently selected from any non-uracil-containing nucleotide; n is an integer from 1 to 4; and p is an integer greater than 4; and a pharmaceutically acceptable carrier. Each of the preferred uracil-containing oligonucleotides set forth above may be present in a composition of this invention. Other preferred oligonucleotides that may be present in the compositions of this invention are oligonucleotides comprising a nucleotide sequence selected from polyUo15, polyUo21, or polydUo21 and oligonucleotides consisting of a nucleotide sequence selected from polyUo15, polyUo21, or polydUo21.

It has also been found that the efficacy of the present oligonucleotide compositions can be enhanced by complexing the oligonucleotide with a secondary compound capable of enhancing the oligonucleotide's stability or ability to enter cells. Thus, in a preferred embodiment, the composition comprises an oligonucleotide complexed to a cationic compound such as PEI or a cationic liposome. In a particularly preferred embodiment, the cationic compound is PEI.

In another aspect, the present invention provides a method of enhancing TLR7-mediated signaling in a cell, the method comprising contacting said cell with an oligonucleotide or composition of the invention. In a preferred embodiment, the method is used in vivo to enhance TLR7-mediated signaling in a subject and the oligonucleotide or composition of this invention is administered to the patient. In another embodiment, the cell in which TLR7-mediated signaling is enhanced is an immune cell. In another embodiment, the cell is a dendritic cell, a B-cell or a monocyte, each of which express TLR7. In another embodiment, the dendritic cell is a plasmacytoid dendritic cell (PDC). In another embodiment, the stimulation of the TLR7 receptor results in the activation of the cell. In another embodiment, the cell is a mouse cell. In another embodiment, the cell is a human cell. In another embodiment, the cell is isolated from a patient with cancer or an infectious disease. In another embodiment, the cell naturally expresses TLR7. In another embodiment, the cell comprises an expression vector whose presence leads to the expression of TLR7 in the cell.

In another embodiment, the method further comprises a step in which the activation of the cell is detected subsequent to said contacting step. In another embodiment, the activation is detected by examining the level of production by the cell of a cytokine selected from the group consisting a type I interferon, for example IFNα, IP-10, IL-8, RANTES, IFNgamma, IL-6, and IL-12 p40. In another embodiment, the examining step is carried out using ELISA. In one embodiment, in a method of identifying or characterizing a candidate TLR7 agonist, ratios of IFN-alpha to IL-6 are detected, and oligonucleotides with the greatest ratio of IFN-alpha:IL-6 are selected as candidate TLR7 agonists.

In another aspect, the present invention provides a method of stimulating an immune response in a patient, the method comprising administering to the patient a pharmaceutical composition comprising any of the herein-described oligonucleotides, and a pharmaceutically-acceptable carrier.

In one embodiment, the patient has cancer or an infectious disease. In another embodiment, the infectious disease is a viral infection. In another embodiment, the administration of the composition results in the stimulation of plasmacytoid dendritic cells (PDC), B-cells or monocytes in the patient.

In another embodiment, the method further comprises a step in which immune cell activity is detected in the patient following the administering step, wherein a detection of increased immune cell activity indicates that the administration is efficacious. In another embodiment, the activity is detected by examining the activity of plasmacytoid dendritic cells (PDC), B-cells or monocytes in said patient. In another embodiment, the activity of the cells is detected by examining the level of expression of a cytokine selected from the group consisting of a type I interferon, for example IFNα, IP-10, IL-8, RANTES, IFNγ, IL-6, and IL-12 p40. In another embodiment, the examining step is carried out using ELISA. In a preferred embodiment, the TLR7 agonist oligonucleotide of the invention induces the expression or secretion of IFNα but does not substantially induce the expression of IL-6.

In another aspect, the present invention provides isolated single-stranded oligonucleotides, compositions which comprise them and methods for optimally stimulating TLR-mediated signaling, through the TLR8 receptor. These oligonucleotides, compositions and methods described herein are useful for enhancing the activation of TLR8-expressing cells, e.g. human dendritic cells, such as myeloid dendritic cells, and certain subsets of regulatory T-cells, in vitro and in vivo. Such oligonucleotides, compositions and methods are useful in a number of clinical applications, including as pharmaceutical agents and methods for treating or preventing conditions such as cancer or infectious diseases, particularly viral infections. These oligonucleotides and compositions of the invention can also be used in methods for assessing the effects of other compounds on TLR8 activity, e.g., in assays to identify or characterize other candidate modulators of TLR8 or of TLR8-expressing cells. The oligonucleotides and compositions are also useful in methods of inducing IFNα production and/or release, particularly by dendritic cells; and in blocking the immunosuppressive activity of CD4⁺ regulatory T cells.

The TLR8 agonist oligonucleotides are based on studies presented herein on TLR7 agonists and elsewhere that demonstrate the TLR7 and TLR8 agonist activity of G, U-rich RNA oligonucleotides (U.S. patent application Ser. No. 0030232074; and Heil, F. et al., (2004) Science 303, pp. 1526-29), and the ability of various phosphorothioate-bonded deoxyguanosine-containing oligonucleotides to agonize TLR8 in CD4⁺ regulatory T cells (Peng G., et al. (2005) Science 309, pp. 1380-1384). Accordingly, in one embodiment, the present invention provides a single-stranded oligonucleotide consisting of between 11 and 50 nucleotides and comprising a sequence selected from: GGG-(X)ₙ-GGG, GG-X-GG-X-GG, or Z(G)ₚZ, wherein each G is independently selected from a guanine-containing nucleotide; each X is independently selected from any nucleotide; each Z is independently selected from any non-guanine nucleotide; n is an integer from 1 to 4; and p is an integer greater than 4, wherein said oligonucleotide comprises at least one non-guanine-containing nucleotide or at least one non-natural linkage.

In one preferred embodiment each of the nucleotides in said oligonucleotide is a guanine-containing oligonucleotide and said oligonucleotide comprises at least one non-natural backbone bond. More preferably, each nucleotide is guanosine or deoxyguanosine.

In another preferred embodiment, said oligonucleotide comprises a sequence selected from (GGG(X)ₙ)ₘ, wherein m is an integer greater than two. Preferably, m is 3 or 4. More preferably, each G is of guanosine. Even more preferably, each n is 1.

In another preferred embodiment, the oligonucleotide comprises the sequence Z(G)ₚZ; and p is an integer greater than 9. Even more is when each G is guanosine, or each G is deoxyguanosine.

According to another preferred embodiment, the oligonucleotide comprises the sequence:

GGXGGXGGXGGXGG.

Also, the presence of one or more of non-natural linkages are preferred in any of the oligonucleotides described above. In a preferred embodiment, at least one non-natural linkage is a phosphorothioate linkage.

In one embodiment, all of the nucleotides in the oligonucleotide are ribonucleotides. In another embodiment, all of the nucleotides in the oligonucleotide are deoxyribonucleotides. In another embodiment, the length of the oligonucleotide is between 11 to 30 nucleotides. In another embodiment, the oligonucleotide is between 21 and 30 nucleotides in length. In yet another embodiment, the oligonucleotide is between 15 and 21 nucleotides in length. In another embodiment, a majority of guanine-containing nucleotides within the oligonucleotide are adjacent to at least one other guanine-containing oligonucleotide.

In yet another embodiment, the oligonucleotide additionally comprises at least one CG doublet, wherein C is an unmethylated cytosine-containing nucleotide, and G is a guanine-containing nucleotide. The CG doublet may be present as part of a sequence selected from GGG-(X)ₙ-GGG, GG-X-GG-X-GG, or Z(G)ₚZ, or outside of those sequences in the oligonucleotide. In an alternate embodiment, the oligonucleotide specifically excludes any CG doublets.

In another embodiment, the TLR8 agonist oligonucleotide further comprises a sequence selected from UUU-(X)ₙ-UUU, UU-X-UU-X-UU, or Y(U)ₚY. Such uracil-containing sequences may overlap the guanine-containing sequences or be completely separate in the oligonucleotide. Such an oligonucleotide will agonize TLR7 as well as TLR8, which is useful in certain human therapeutic and other uses.

The present invention also provides a composition comprising an isolated single stranded oligonucleotide of between 11 and 50 nucleotides in length, and comprising a sequence selected from: GGG-(X)ₙ-GGG, GG-X-GG-X-GG, or Z(G)ₚZ, wherein each G is independently selected from a guanine-containing nucleotide; each X is independently selected from any nucleotide; each Z is independently selected from any non-guanine nucleotide; n is an integer from 1 to 4; and p is an integer greater than 4. Each of the preferred guanine nucleotide-containing oligonucleotides set forth above may be present in a composition of this invention. Other preferred oligonucleotides that may be present in the compositions of this invention are oligonucleotides consisting entirely of guanine-containing nucleotides that are bound to one another via phosphodiester bonds.

In a preferred embodiment, the composition comprises an oligonucleotide complexed to a cationic compound such as PEI or a cationic liposome. In a particularly preferred embodiment, the cationic compound is PEI.

In another aspect, the present invention provides a method of enhancing TLR8-mediated signaling in a cell, the method comprising contacting said cell with an oligonucleotide or composition of the invention. In a preferred embodiment, the method is used in vivo to enhance TLR8-mediated signaling in a subject and the oligonucleotide or composition of this invention is administered to the patient. In another embodiment, the cell in which TLR8-mediated signaling is enhanced is an immune cell. In another embodiment, the cell is a dendritic cell. In another embodiment, the cell is a CD4⁺ regulatory T-cell. In another embodiment, the stimulation of the TLR8 receptor results in the activation of the cell. In another embodiment, the stimulation of the TLR8 receptor results in the deactivation of a CD4⁺ regulatory T-cell. In another embodiment, the cell is a mouse cell. In another embodiment, the cell is a human cell. In another embodiment, the cell is isolated from a patient with cancer or an infectious disease. In another embodiment, the cell naturally expresses TLR8. In another embodiment, the cell comprises an expression vector whose presence leads to the expression of TLR8 in the cell.

In another embodiment, the method further comprises a step in which the activation of the cell is detected subsequent to said contacting step. In another embodiment, the activation is detected by examining the level of production by the cell of a cytokine selected from the group consisting of a type I interferon, for example IFNα, IP-10, IL-8, RANTES, IFNgamma, IL-6, and IL-12 p40. In another embodiment, the examining step is carried out using ELISA.

In another embodiment, the method further comprises a step in which the deactivation of a CD4⁺ regulatory T-cell is detected subsequent to said contacting step. In another embodiment, the deactivation is detected by determining the ability of the CD4⁺ regulatory T-cells to suppress naïve CD4⁺ T cell proliferation. In another embodiment, the examining step is carried out by detecting [³H]thymidine incorporation into naïve CD4⁺ T cells incubated with CD4⁺ regulatory T-cells.

In another aspect, the present invention provides a method of stimulating an immune response in a patient, the method comprising administering to the patient a pharmaceutical composition comprising any of the herein-described oligonucleotides, and a pharmaceutically-acceptable carrier.

In one embodiment, the patient has cancer or an infectious disease. In another embodiment, the infectious disease is a viral infection. In another embodiment, the administration of the composition results in the stimulation of dendritic cells in the patient.

In another embodiment, the method further comprises a step in which immune cell activity is detected in the patient following the administering step, wherein a detection of increased immune cell activity indicates that the administration is efficacious. In another embodiment, the activity is detected by examining the activity of dendritic cells in said patient. In another embodiment, the activity of the cells is detected by examining the level of expression of a cytokine selected from the group consisting of a type I interferon, for example IFNα, IP-10, IL-8, RANTES, IFNgamma, IL-6, and IL-12 p40. In another embodiment, the examining step is carried out using ELISA. In another embodiment, the activity is detected by examining the activity of CD4⁺ regulatory T-cells in said patient.

### BRIEF DESCRIPTION OF THE DRAWINGS

FIG. 1 shows that PolyU RNA 21-mer oligonucleotides induce IFNα by Flt3L-DC irrespective of phosphodiester or phosphorothioate bonds. Bulk cultures of C57BL/6 Flt3L-DC were stimulated with different doses of RNA and IFNα levels in supernatants were measured by ELISA after overnight culture (triplicate samples±1 SD). (A) Complexes of PEI with homopolymeric polyU phosphodiester RNA of undefined length were compared to PEI complexes with 21-mer polyU RNA phosphodiester (polyUo-21) and phosphorothioate (polyUs-21) oligonucleotides. Phosphodiester (B) or phosphorothioate (C) polyU RNA 21-mer were used for stimulation of Flt3L-derived BM-DC in form of complexes with PEI (+PEI) or as free oligonucleotides (w/o PEI). The RNA concentration is depicted in μg/ml rather than in μmolar, since the average molecular weight of the polyU preparation is not known. Data are representative of at least three independent experiments.

FIG. 2 shows that PolyU RNA oligonucleotide mediated induction of IFNα in Flt3L-DC cultures correlates with the size of the oligonucleotides. Bulk cultures of C57BL/6 Flt3L-DC were stimulated with different doses of RNA oligonucleotides and IFNα levels in supernatants were measured by ELISA after overnight culture (triplicate samples±1 SD). Complexes of PEI with 21-mer, 15-mer and 10-mer polyU phosphodiester (A) or phosphorothioate (B) RNA oligonucleotides were used for stimulation of cells. Concentration of RNA is depicted in μmolar to normalize for the molecular weight of the different oligonucleotides. Data are representative of at least three independent experiments.

FIG. 3 shows that backbone modifications affect the IFNα stimulatory activity of polyU oligonucleotides. Bulk cultures of C57BL/6 Flt3L-DC were stimulated with different doses of 21-mer RNA oligonucleotide/PEI complexes and IFNα levels in supernatants were measured by ELISA after overnight culture (triplicate samples±1 SD). (A) polyU phosphodiester RNA oligonucleotide (polyUo-21) was compared to polyU phosphodiester DNA oligonucleotide (polydUo-21). (B) Similarly, polyU phoshorothioate RNA (polyUs-21) and DNA (polydUs-21) oligonucleotides were used for stimulation of Flt3L-DC. (C) Cells were treated with polyU RNA oligonucleotides containing phosphorothioate (polyUs-21) or 2′-O-methyl (polyUm-21) backbone modifications. Data are representative of at least three independent experiments.

FIG. 4 shows that the stimulatory activity of RNA oligonucleotides correlates with the number of uridine moieties they contain and double and triple uridine moieties are more stimulatory than single uridine moieties. (A-D) Bulk cultures of C57BL/6 Flt3L-DC were stimulated with different doses of 21-mer RNA oligonucleotide/PEI complexes and IFNα levels in supernatants were measured by ELISA after overnight culture (triplicate samples±1 SD). PolyU phoshorothioate RNA oligonucleotide (polyUs-21) served as reference TLR7 ligand in all experiments. For composition of different 21-mer oligonucleotides see Table 1. Data are representative of at least three independent experiments.

FIG. 5 shows that neither polyA, polyC, polyT RNA oligonucleotides nor ribospacer moieties induce IFNα induction in Flt3L-DC. (A, B) Bulk cultures of C57BL/6 Flt3L-DC were stimulated with different doses of 21-mer RNA oligonucleotide/PEI complexes and IFNα levels in supernatants were measured by ELISA after overnight culture (triplicate samples±1 SD). (A) PolyU (polyUs-21), polyA (polyAs-21), polyC (polyCs-21) and polyT (polyTs-21) phoshorothioate RNA oligonucleotides were used for stimulation of Flt3L-DC. (B) IFNα induction by homopolymeric polyU oligonucleotide was compared to IFNα induction by composite oligonucleotides containing a mixture of uridine and cystidine moieties (SSD13), uridine and ribospacer moieties (polyUspacer) or cystidine and ribospacer moieties (polyCspacer). For composition of different 21-mer oligonucleotides see Table 1. Data are representative of at least three independent experiments.

FIG. 6 provides a schematic representation of the molecular structure of RNA nucleotides and the nucleotide analogues loxoribine and R848. (A) Depiction of a dimer consisting of uridine RNA nucleotides. The backbone modifications (DNA versus RNA, 2′-O-methyl and phosphorothioate modifications) that have been tested are indicated in blue and the organic base is highlighted in grey. (B) Schematic representation of the organic base structure of the purines cytosine and thymine highlighted in grey. (C) Depiction of the molecular structure of R848, loxoribine and a uridine nucleotide. The moieties that are shared between the structures of these three molecules and that are indicated to play a role in the recognition of these ligands by TLR7 are highlighted in grey.

FIG. 7 shows that polyU RNA oligonucleotides are strong inducers of IFNα from plasmacytoid DC unlike the TLR7 ligands loxoribine and R848, which are better in inducing IL-6. Bulk cultures of C57BL/6 Flt3L-DC were stimulated with different TLR7 ligands and with the DNA oligonucleotide CpG 1668 (0.5 μg/ml) stimulating TLR9. TLR7 ligands used were the RNA oligonucleotide polyUs-21 (1 μg/ml) complexed to PEI and the imidazoquinolins loxoribine (100 mM) and R848 (10 μg/ml). All TLR ligands were used at doses, which induce maximum levels of cytokine production by plasmacytoid DC. IFNα (A) and IL-6 (B) levels in supernatants were measured by ELISA after overnight culture (triplicate samples±1 SD). Data are representative of at least three independent experiments.

FIG. 8 shows that TLR7 ligands induce IFNα and IL-6 in human plasmacytoid DC. (A) Cultures of human pDC were stimulated with different doses (expressed in μmolar) of polyUs 21-mer versus 1 μM of polyAs 21-mer, both as PEI complexes. IFNα levels in supernatants were measured by ELISA after overnight culture. (B) Cultures of human pDC were stimulated with polyUs 21-mer (10 μM) as PEI complexes versus polyAs 21-mer (10 μM) as PEI complexes, RNA9.2DR (1 μM) as LyoVec complexes and R848 (1 μM). Levels of IFNα (B) and IL-6 (C) in supernatants were measured by ELISA after overnight culture. Data are the mean of triplicate samples±1 SEM and are representative of at least three independent experiments.

Table 1 provides a list of oligonucleotides tested. Phosphodiester bonds (Uo), phoshorothioate bonds (Us, As, Cs, Gs, Ts), 2′-O-methyl modification (Um) and DNA oligos (dUs, dUo).

### DETAILED DESCRIPTION OF THE INVENTION

### Definitions

Unless otherwise defined, all technical and scientific terms used herein have the same meaning as commonly understood by one of ordinary skill in the art to which this invention belongs.

As used herein, the term “antigen” refers to any molecule capable of being recognized by a T-cell antigen receptor or B-cell antigen receptor. The term broadly includes any type of molecule which is recognized by a host immune system as being foreign. Antigens generally include but are not limited to cells, cell extracts, proteins, polypeptides, peptides, polysaccharides, polysaccharide conjugates, peptide and non-peptide mimics of polysaccharides and other molecules, small molecules, lipids, glycolipids, polysaccharides, carbohydrates, viruses and viral extracts, and multicellular organisms such as parasites, and allergens. With respect to antigens that are proteins, polypeptides, or peptides, such antigens can include nucleic acid molecules encoding such antigens. Antigens more specifically include, but are not limited to, cancer antigens, which include cancer cells and molecules expressed in or on cancer cells; viral antigens, which include whole and attenuated virus and molecules expressed in or on viruses; and allergens.

As used herein, the terms “Toll-like receptor” and, equivalently, “TLR” refer to any member of a family of at least eleven highly conserved mammalian pattern recognition receptor proteins (TLR1-TLR11) which recognize pathogen-associated molecular patterns (PAMPs) and act as key signaling elements in innate immunity. TLR polypeptides share a characteristic structure that includes an extracellular (extracytoplasmic) domain that has leucine-rich repeats, a transmembrane domain, and an intracellular (cytoplasmic) domain that is involved in TLR signaling. TLRs include but are not limited to human TLRs.

As referred to herein, “Toll-like receptor-7,” or “TLR7,” refers to nucleic acids or polypeptides sharing at least 70%; 80%, 90%, 95%, 96%, 97%, 98%, 99%, or more sequence identity to publicly available TLR7 sequence, e.g., GenBank accession numbers AF240467 or AAF60188, for human TLR7, or GenBank accession numbers AY035889 or AAK62676, for murine TLR7. Derivatives and fragments of any of such sequences are also encompassed. GenBank accession numbers for human TLR7 are provided for AF240467(SEQ ID NO 31) and AAF60188 (SEQ ID NO 32).

As used herein “TLR signaling” refers to an ability of a TLR polypeptide, particularly TLR7 and/or TLR8, to activate the Toll/IL-1R (TIR) signaling pathway, also referred to herein as the TLR signal transduction pathway. Changes in TLR activity can be measured, e.g., by assays designed to measure expression of genes under control of NF-kB-sensitive promoters and enhancers. Such genes can be naturally occurring genes or they can be genes artificially introduced into a cell. Naturally occurring reporter genes include the genes encoding IL-1β, IL-6, IL-8, the p40 subunit of interleukin 12 (IL-12 p40), and the costimulatory molecules CD80 and CD86. Other genes can be placed under the control of such regulatory elements and thus serve to report the level of TLR signaling.

As used herein, the terms “stimulating” or “activating” with respect to the effect of the herein-described oligonucleotides on TLR7 or TLR8 refers to the ability of the oligonucleotide to bind, directly or indirectly, to TLR7 or TLR8 present on the surface or in a cytoplasmic compartment of a cell, e.g., endosome surface, and to induce TLR signaling. Any detectable difference in TLR signaling can indicate that an oligonucleotide stimulates or activates a TLR7 or TLR8 receptor. Signaling differences can be manifest in any of a number of ways, including changes in the expression of target genes, in the phosphorylation of signal transduction components, in the intracellular localization of downstream elements such as NK-kB, in the association of certain components (such as IRAK) with other proteins or intracellular structures, or in the biochemical activity of components such as kinases (such as MAPK). Regardless of the assay used, an alteration of 5%, 10%, 20%, 30%, 40%, 50%, 60%, 70%, 80%, 90%, 100%, 200%, 300%, 400%, 500%, 1000%, or more in any aspect of TLR signaling is indicative of stimulation or activation.

The term “activate a cell” as used herein, means causing the cell to increase expression of one or more cytokine selected from the group consisting of IFNα, IL-6, and IL-12 p40.

As used herein, an “effective amount” refers to any amount that is necessary or sufficient for achieving or promoting a desired outcome. In some instances an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “immune cell” refers to a cell belonging to the immune system. Immune cells include T lymphocytes (T cells), B lymphocytes (B cells), natural killer (NK) cells, granulocytes, neutrophils, macrophages, monocytes, dendritic cells, and specialized forms of any of the foregoing, e.g., plasmacytoid dendritic cells, plasma cells, NKT, T helper, regulatory T cells, gamma delta T cells and cytotoxic T lymphocytes (CTL).

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; thyroid cancer; as well as other carcinomas and sarcomas. Cancers can be primary or metastatic.

As used herein, the terms “infection” and, equivalently, “infectious disease” refer to a condition in which an infectious organism or agent is present in a detectable amount in the blood or in a normally sterile tissue or normally sterile compartment of a subject. Infectious organisms and agents include viruses, bacteria, fungi, and parasites. The terms encompass both acute and chronic infections, as well as sepsis.

As used herein, the term “innate immune response” refers to any type of immune response to certain pathogen-associated molecular patterns (PAMPs). Innate immunity, which is also known in the art as natural or native immunity, involves principally neutrophils, granulocytes, mononuclear phagocytes, dendritic cells, NKT cells, and NK cells. Innate immune responses can include, without limitation, type I interferon production (e.g., IFN-alpha), neutrophil activation, macrophage activation, phagocytosis, opsonization, complement activation, and any combination thereof.

As used herein, “cytokine” refers to any of a number of soluble proteins or glycoproteins that act on immune cells through specific receptors to affect the state of activation and function of the immune cells. Cytokines include interferons, interleukins, tumor necrosis factor, transforming growth factor beta, colony-stimulating factors (CSFs), chemokines, as well as others. Various cytokines affect innate immunity, acquired immunity, or both. Cytokines specifically include, without limitation, IFN-α, IFN-β, IFN-γ, IL-1, IL-2, IL-3, IL-4, IL-5, IL-6, IL-9, IL-10, IL-12, IL-13, IL-18, TNF-α, TGF-β, granulocyte colony-stimulating factor (G-CSF), and granulocyte-macrophage colony-stimulating factor (GM-CSF). Chemokines specifically include, without limitation, IL-8, IP-10, I-TAC, RANTES, MIP-1α, MIP-1β, Gro-α, Gro-β, Gro-γ, MCP-1, MCP-2, and MCP-3.

As used herein, the terms “treat,” “therapy,” or “therapeutic,” as used in reference to a disorder, disease, or condition means to intervene in such disorder, disease, or condition in a way that is designed to prevent or slow the development of, to prevent, slow or halt the progression of, or to eliminate the disorder, disease, or condition. It will be appreciated that the disorder, disease, or condition need not in fact be halted or eliminated for a method to be considered a treatment or therapy. The terms “prevent” and “prophylactic” with respect to a disorder, disease, or condition are related to treatment, but are used with individuals who are at risk of developing the disorder, disease, or condition, but who do not show any signs or symptoms at the time of administration.

The terms “nucleic acid” and “oligonucleotide” are used interchangeably to mean a chain of multiple nucleotides bound to one another. The term “nucleotide” as used herein means a molecule comprising a sugar linked to a phosphate group and to an exchangeable organic base. A nucleotide in the oligonucleotides of this invention can be modified at the sugar, phosphate and/or base moiety. The sugar moiety may be a ribose, deoxyribose or arabinose, preferably a ribose or a deoxyribose, and more preferably a ribose. The sugar may also comprise other modifications at the 2′ position (e.g., 2′-O-methyl modifications, 2′-O-methoxyethyl modifications, 2′-amino modifications, 2′-deoxy modifications, 2′-halo modifications such as 2′-fluoro; combinations of the above, such as 2′-deoxy-2′-fluoro modifications) on those nucleotides. However, such other 2′ sugar modifications are limited to nucleotides that are not crucial for TLR agonism. Thus a 2′ sugar modifications is not present on any uracil-containing nucleotide in a U-rich region or any guanine-containing nucleotide in a G-rich region of the oligonucleotides of this invention. More preferably, a 2′ sugar modification is not present on any nucleotide in a U-rich or G-rich region of the oligonucleotide. Nucleic acid molecules can be obtained from existing nucleic acid sources (e.g., genomic or cDNA), but are preferably synthetic (e.g., produced by nucleic acid synthesis).

The base portion of a nucleotide is a purine or a pyrimidine. Purines and pyrimidines include but are not limited to naturally occurring bases, such as adenine, cytosine, guanine, thymidine and uracil, and chemically modified bases, such as inosine, 2,4-diaminopurine; 2,6-diaminopurine; 2-alkyl adenine; 2-alkyl inosine; 2-amino purine; 2-amino-6-chloropurine; 2-halo purine; 2-thiocpyrimidine; 4-thiouracil; 5-(C1-C6)-alkyl pyrimidine; 5-(C2-C6)-alkenyl pyrimidine; 5-(C2-C6)-alkynyl pyrimidine; 5-(hydroxymethyl)uracil; 5-amino pyrimidine; 5-halo pyrimidine; 5-hydroxy pyrimidine; 5-hydroxymethylpyrimidine; 6-azo pyrimidine; 6-methyl purine; 7-deazapurine; 7-methyl purine; 8-azapurine; other 8-substituted purines; dihydrouracil; hypoxanthine; N2-dimethyl purine; pseudouracil; substituted 7-deazapurine; and xanthine. This list is meant to be exemplary and is not to be interpreted to be limiting.

The phosphate group on a nucleotide present in an oligonucleotide of this invention may be modified by another phosphorus containing moiety capable of binding to another nucleotide. Such modified groups include a phosphoramidate, a phosphorothioate, and a phosphorodithioate. A bond formed between nucleotides in the oligonucleotides of this invention by any bonds other than phosphodiester bonds is termed a “non-natural linkage.”

Other modifications are those that may be present on the 3′ or 5′ terminal nucleotide of an oligonucleotide of this invention, and include a 3′-and/or 5′-terminal cap, a terminal 3′-5′ linkage, and a 5′-terminal phosphate group or modified phosphate group.

Examples of a 5′-cap includes, but is not limited to, glyceryl, inverted deoxy abasic residue (moiety); 4′,5′ methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5 dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4 dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4 butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

The term “uracil-containing nucleotide” as used herein encompasses uracil and deoxyuracil and any nucleotide containing a modified uracil. The term “non-uracil-containing nucleotide” as used herein means any nucleotide that does not comprise uracil or a modified uracil as a base. The term “non-guanine-containing nucleotide” as used herein means any nucleotide that does not comprise guanine or modified guanine as a base.

As used herein, a coding sequence and a gene expression sequence are said to be operably linked when they are covalently linked in such a way as to place the expression or transcription and/or translation of the coding sequence under the influence or control of the gene expression sequence. Two DNA sequences are said to be operably linked if induction of a promoter in the 5′ gene expression sequence results in the transcription of the coding sequence and if the nature of the linkage between the two DNA sequences does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a gene expression sequence would be operably linked to a coding sequence if the gene expression sequence were capable of effecting transcription of that coding sequence such that the resulting transcript is translated into the desired protein or polypeptide.

The present invention provides novel oligonucleotides, compositions and methods for stimulating immune responses. The present invention is based on studies involving the systematic analysis of different oligonucleotides with respect to their ability to stimulate TLR-mediated signaling, particularly through dendritic cells such as plasmacytoid dendritic cells, and specifically through the TLR7 receptor.

Oligonucleotides, compositions and methods described herein are useful for enhancing immune stimulation in vitro and in vivo. Such oligonucleotides, compositions and methods thus will find use in a number of clinical applications, including as pharmaceutical agents and methods for treating or preventing conditions such as cancer or infectious diseases, particularly viral infections. The oligonucleotides and compositions of the invention can also be used in methods for the preparation of medicaments for use in the treatment of such conditions. The compositions of the invention were up to approximately 30 times more potent in inducing IFNα by Flt3L-DC than the low molecular weight anti-viral compounds R848 and loxoribine which have also been reported to act via TLR7 and TLR8. It will be appreciated that the herein-described oligonucleotides can also be used in assays for identifying modulators of TLR7 or TLR8, e.g., activators or inhibitors of TLR7 or TLR8 signaling or of TLR7- or TLR8-expressing cells.

The present invention is based on the surprising discovery that the nucleotide uridine or deoxyuridine is the essential controlling element in determining whether or not an oligonucleotide can activate TLR7. Accordingly, even short oligonucleotides, that comprise sufficient uracil-containing oligonucleotides, either in terms of the absolute number of uridines or in terms of their grouping within the oligonucleotide, can be used to effectively stimulate TLR7 receptors in vivo or in vitro.

### The Oligonucleotides of the Invention

In one general embodiment, the invention provides a single-stranded oligonucleotide consisting of between 10 and 50 nucleotides (e.g., comprising 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides; preferably between 10 and 19 nucleotides in length, between 15 and 30 nucleotides in length, between 19 and 50 nucleotides in length, between 15 and 21 nucleotides in length, or between 21 and 30 nucleotides in length; more preferably 15 or 21 nucleotides in length, and most preferably 21 nucleotides in length); and comprising a sequence selected from: UUU-(X)ₙ-UUU, or UU-X-UU-X-UU, or Y(U)ₚY, wherein each U is independently selected from a uracil-containing nucleotide; each X is independently selected from any nucleotide; n is an integer from 1 to 4; and p is an integer greater than 4; and wherein said oligonucleotide comprises at least one non-uracil-containing nucleotide or at least one non-natural linkage. Such an oligonucleotide will demonstrate an enhanced ability to stimulate TLR7. Preferably the oligonucleotides of the invention comprises a stretch of at least 6, 7, 8, 9, 10, 11, 12, 13, 14, or 15 consecutive uridines.

In one preferred embodiment, said oligonucleotide comprises the sequence UUU-(X)ₙ-UUU, and n is 1. In another preferred embodiment, the oligonucleotide comprises the sequence (UUU-(X)ₙ)ₘ, wherein n is an integer from 1 to 4, more preferably 1; and m is an integer greater than 2, preferably 3 or 4. In another preferred embodiment, said oligonucleotide comprises the sequence Y(U)ₚY, and p is an integer greater than 9. In each of these described embodiments, it is preferred that each U is uridine.

Particularly preferred are a 21-mer oligonucleotide with one or more phosphorothioate linkages consisting entirely of uridines (e.g., polyUs21); a 21-mer comprising a stretch of at least 10 consecutive uridines (e.g., SSD30); and a 21-mer comprising the sequence UUXUUXUUXUUXUU, wherein each U is uridine and each X is independently selected from any nucleotide (e.g., SSD 28).

Generally, the greater the proportion of uracil-containing nucleotides present within an oligonucleotide, the greater its ability to stimulate TLR7. Accordingly, in one preferred embodiment, the single-stranded oligonucleotide comprises at least 50%, 60%, 70%, 80%, 90%, 95%, 96%, 97%, 98%, or 99% uracil-containing nucleotides. Preferably, each of the uracil-containing nucleotides is uridine. In one particularly preferred embodiment, the oligonucleotide is made up entirely of uridines and comprises at least one non-natural linkage.

Other preferred oligonucleotides include oligonucleotides, optionally with one or more phosphorothioate linkages, between 10 and 50 nucleotides in length, and comprising a stretch of at least 10 consecutive uridines; and an oligonucleotide comprising the sequence UUXUUXUUXUUXUU, wherein each U is uridine and each X is independently selected from any nucleotide other than G (guanine), other than C, or other than G and C.

It has also been discovered that the guanine content of the oligonucleotides is generally not important for TLR7 activity. Accordingly, in one embodiment, the present oligonucleotides can contain less than 50%, 40%, 30%, 20%, 10%, or 5% guanine-containing nucleotides.

A number of oligonucleotides that follow the present teaching and which are thus capable of stimulating TLR7 are shown in Table 1, particularly polyUo-21, polyUo-15, polyUo-10, polyUs-21, polyUs-15, polydUo-21, polydUs-21, SSD8, SSD9, SSD10, SSD13, SSD14, SSD15, SSD21, SSD22, SSD23, SSD24, SSD28, SSD29, and SSD30. Any of these oligonucleotides, or variants, derivatives, or longer oligonucleotides comprising any of these oligonucleotides, can be used.

In another general embodiment, the invention provides a single stranded oligonucleotide consisting of between 11 and 50 nucleotides (e.g., comprising 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides; preferably between 10 and 19 nucleotides in length, between 15 and 30 nucleotides in length, between 19 and 50 nucleotides in length, between 15 and 21 nucleotides in length, or between 21 and 30 nucleotides in length; more preferably 15 or 21 nucleotides in length, and most preferably 21 nucleotides in length); and comprising a sequence selected from: GGG-(X)ₙ-GGG, GG-X-GG-X-GG, or Z(G)ₚZ, wherein each G is independently selected from a guanine-containing nucleotide; each X is independently selected from any nucleotide; each Z is independently selected from any non-guanine nucleotide; n is an integer from 1 to 4; and p is an integer greater than 4, wherein said oligonucleotide comprises at least one non-guanine-containing nucleotide or at least one non-natural linkage. These G-rich oligonucleotides will display enhanced ability to agonize TLR8.

In one preferred embodiment, said oligonucleotide comprises the sequence GGG-(X)ₙ-GGG, and n is 1. In another preferred embodiment, the oligonucleotide comprises the sequence (GGG-(X)ₙ)ₘ, wherein n is an integer from 1 to 4, more preferably 1; and m is an integer greater than 2, preferably 3 or 4. In another preferred embodiment, said oligonucleotide comprises the sequence Z(G)ₚZ, and p is an integer greater than 9. In each of these described embodiments, it is preferred that each G is guanosine.

It will be appreciated that the herein-described oligonucleotides can contain nucleotides other than those imparting TLR7- or TLR-8 stimulation ability. For example, the TLR7-activating sequences (e.g., sequences shown in Table 1) or TLR-8 activating sequences can be present in an oligonucleotide together with other sequence elements, e.g., short sequences designed to enhance stability, to direct targeting to specific cells or intracellular compartments, to enhance binding by various proteins, etc. In one embodiment, an oligonucleotide of this invention will additionally comprise one or more CpG dinucleotides and be able to agonize TLR9 as well as either TLR7 or TLR8. In another embodiment, an oligonucleotide of this invention will comprise both TLR-7 and TLR-8 activating sequences (i.e., a) a sequence selected from UUU-(X)ₙ-UUU, or UU-X-UU-X-UU, or Y(U)ₚY; and b) a sequence selected from GGG-(X)ₙ-GGG, GG-X-GG-X-GG, or Z(G)ₚZ, wherein each X, n and p is independently selected).

So long that the herein-described sequence features are fulfilled, the present oligonucleotides are relatively flexible in terms of the backbone linking the nucleotides together. Modifying the phosphate backbone of the oligonucleotides, for example, can enhance their stability in vitro while maintaining TLR7-stimulating and/or TLR-8 stimulating activity. In a preferred embodiment, the oligonucleotide comprises at least one phosphorothioate linkage. In a particularly preferred embodiment, all the linkages are phosphorothioate. Other modified nucleic acids include, inter alia, alkylphosphonate, arylphosphonate, alkylphosphorothioate, arylphosphorothioate, methylphosphonate, methylphosphorothioate, phosphorodithioate, p-ethoxy, morpholino, and combinations thereof.

In another example, an oligonucleotide may optionally specifically exclude a sequence (G)ₚ wherein p is 1, 2, 3, 4, 5, 6, 7, 8, 9 or 10, optionally wherein each G is a deoxyribonucleotide. In another example, an oligonucleotide may optionally specifically exclude a sequence (U)ₚ wherein p is 1, 2, 3, 4, 5, 6, 7, 8, 9 or 10, optionally wherein each U is a ribonucleotide. In another example, an oligonucleotide may optionally specifically exclude a sequence selected from the group consisting of CUGU, UUGU, CUUU, UUUU, GUUGUUUU, and GUUGU, optionally wherein each nucleotide is a ribonucleotide.

In numerous embodiments, the oligonucleotides will be formulated along with other components. For example, in one preferred embodiment, the oligonucleotide is complexed with a cationic substance such as PEI. Such compounds can help protect the oligonucleotides against degradation and also facilitate their uptake into cells in vitro or in vivo. In other embodiments, one or more oligonucleotides will be formulated with a pharmaceutical carrier, in preparation for its use in a clinical setting.

Synthesis of oligonucleotides having specific sequences and comprising backbone and/or base modifications is well known in the art and easily carried out. For example, oligonucleotides comprising any desired sequence and including any of a large number of backbone or base modifications can be prepared using automated synthesizers or ordered from commercial suppliers. Generally, the nucleic acids of the invention can be synthesized de novo using the β-cyanoethyl phosphoramidite method (Beaucage S L et al. (1981) Tetrahedron Lett 22:1859); or the nucleoside H-phosphonate method (Garegg et al. (1986) Tetrahedron Lett 27:4051-4; Froehler et al. (1986) Nucl Acid Res 14:5399-407; Garegg et al. (1986) Tetrahedron Lett 27:4055-8; Gaffney et al. (1988) Tetrahedron Lett 29:2619-22).

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Pat. No. 092,574) can be prepared by automated solid phase synthesis using commercially available reagents. Methods for making other DNA backbone modifications and substitutions have been described. Uhlmann E et al. (1990) Chem Rev 90:544; Goodchild J (1990) Bioconjugate Chem 1:165.

### Assaying the Ability of the Oligonucleotides to Stimulate TLR7 and TLR8

The oligonucleotides of the present invention can be assessed in vitro for their ability to stimulate TLR7 in pDCs or in other cell types using any of a variety of assays. Such assays can be used, inter alia, for testing derivatives of the herein-provided sequences or for assessing novel sequences designed according to the teachings of the present specification for their ability to stimulate TLR7. Such assays can also be used to identify other modulators of TLR7-expressing cells, e.g., using the herein-described oligonucleotides as standards or controls. In vitro stimulation of pDCs are also useful, e.g., for evaluating pDCs or other TLR7-expressing cells from an individual. For example, pDCs can be removed from a patient with cancer or an infectious disease, and the ability to stimulate the pDCs using the present oligonucleotides assessed. A detection that pDCs can be stimulated in such assays indicates that the patient is a suitable candidate for therapeutic or prophylactic methods involving the administration of the present oligonucleotides.

Oligonucleotides can be assessed in vitro for their ability to stimulate TLR8 in myeloid dendritic cells (mDCs), monocytes, or CD4⁺, CD25⁺ regulatory T (“Treg”) cells, or in other cell types using a similar variety of assays. In vitro stimulation of these cell types is also useful for evaluating the ability of a patient to be immunostimulated by the oligonucleotides of this invention.

The present in vitro assays can be performed either with isolated cells that naturally express TLR7 or TLR8, or with cells that do not normally express the requisite TLR but into which expression constructs encoding TLR7 and/or TLR8 have been introduced.

In one embodiment the cell naturally expresses functional TLR and is, e.g., for TLR7, a B-cell, monocyte, pDC or other dendritic cell type; and for TLR8, a mDC, a monocyte or a Treg cell. pDCs can be isolated from, e.g., bone marrow, the blood, or spleen using standard methods (see, e.g., Diebold et al. (2004) Science 303:1529; Heil et al. (2004) Science 303/1526; Triantafilou et al. (2005) Eur J Immunol 35:2416; Lee et al. (2003) PNAS 100: 6646; Hornung et al. (2005) Nature Med 11: 263; U.S. Patent application no. US 2003/0232074; the disclosures of each of which are herein incorporated in their entireties). Also, suitable murine cells expressing TLR7 include Flt3L-DCs isolated, e.g., from bone marrow progenitors isolated from C57BL/6, Balb/c, CBA, 129 or other mice, for example as can be obtained from Charles River UK. In humans, suitable cell types expressing TLR7 also include freshly isolated plasmacytoid DC from PBMC. This cell line was established from the peripheral blood of a 61 year old man at the time of diagnosis of multiple myeloma (IgG lambda type) (Matsuoka Y et al. (1967) Proc Soc Exp Biol Med 125:1246-50, the entire disclosure of which is herein incorporated by reference). It is known that RPMI 8226 cells secrete a number of other chemokines and cytokines including IL-8, IL-10 and IP-10 in response to immunostimulatory nucleic acids. The RPMI 8226 cell line has been found to respond to certain small molecules including imidazoquinoline compounds. For example, incubation of RPMI 8226 cells with the imidazoquinoline compound R848 (resiquimod) induces IL-8, IL-10, and IP-10 production. It has recently been reported that R848 mediates its immunostimulatory effects through TLR7 and TLR8.

Myeloid dendritic cells and monocytes for assaying TLR8 may also be isolated from bone marrow, peripheral blood, or fetal tissue. Treg cells are typically isolated from peripheral blood (see, Peng G. et al., (2005), Science 309, p. 1380-84). Since TLR8 is non-functional in mice, only human cell lines are a source of functional TLR8. Such cell lines include TIL102, TIL 164, and THP-1.

Any of a large variety of cell types can be made to express TLR7 or TLR8 for the purposes of the present assays. For example, human 293 fibroblasts (ATCC CRL-1573), which do not express TLR7 or TLR8, can be used. Such cells can be transiently or stably transfected with suitable expression vector (or vectors) so as to yield cells that express TLR7 or TLR8. Such stably transfected HEK-293 cell are commercially available (InvivoGen, San Diego, Calif.). In one embodiment, cells can be used that normally express TLR7 or TLR8, albeit at a significantly lower level than in the presence of the corresponding expression construct. TLR7- or TLR-8 encoding expression constructs can be made using standard molecular biology methods, typically including regulatory sequences capable of constitutively driving expression of operably linked coding sequences, and a coding sequence encoding all or part of TLR7 or TLR8. Such vectors are standard in the art and are described, e.g., in Molecular Cloning: A Laboratory Manual (Sambrook et al.; Cold Spring Harbor Laboratory Press; 3rd edition (Jan. 15, 2001), or Short Protocols in Molecular Biology (Ausubel et al; Current Protocols; 5 edition (Oct. 18, 2002), each of which is incorporated herein by reference in its entirety.

Constitutive mammalian promoters that can be used to drive TLR7 or TLR8 expression include, but are not limited to, the promoters for the following genes: hypoxanthine phosphoribosyl transferase (HPRT), adenosine deaminase, pyruvate kinase, β-actin promoter and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, the long terminal repeats (LTR) of Moloney leukemia virus and other retroviruses, and the thymidine kinase promoter of herpes simplex virus. Other constitutive promoters are known to those of ordinary skill in the art.

The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

Nucleic acid and amino acid sequences for TLR7 and TLR8 from humans and other species are available from public databases such as GenBank. For example, nucleic acid and amino acid sequences for human TLR7 (hTLR7) can be found as GenBank accession numbers AF240467 (coding region spanning nucleotides 135-3285) and AAF60188, respectively. Nucleic acid and amino acid sequences for murine TLR7 (mTLR7) can be found as GenBank accession numbers AY035889 (coding region spanning nucleotides 49-3201) and AAK62676, respectively.

Typically, the TLR-expressing cells will be introduced into a suitable container, e.g. 96-well plates, together with the oligonucleotide and appropriate culture medium. Typically, a candidate oligonucleotide will be tested in parallel at different concentrations to obtain a different response to the various concentrations. Typically, one of these concentrations serves as a negative control, i.e., at zero concentration of agent or at a concentration of agent below the limits of assay detection.

The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C., more typically about 37° C. Incubation times preferably are minimized to facilitate rapid, high throughput screening, and typically are between 1 minute and 48 hours.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

After incubation (for, e.g., 18-20 hours), the activation (or lack thereof) of the cells can be assessed using any of a large number of potential methods. Assays for detecting TLR7 and TLR8 activation are described, inter alia, in Diebold et al. (2004) Science 303:1529; Heil et al. (2004) Science 303/1526; Triantafilou et al. (2005) Eur J Immunol 35:2416; Lee et al. (2003) PNAS 100: 6646; Hornung et al. (2005) Nature Med 11: 263; U.S. Patent application no. US 2003/0232074; the disclosures of each of which are herein incorporated in their entireties.

In a preferred embodiment, the level of TLR7- or TLR8-responsive cytokines is measured in the culture medium following incubation of the cells with the oligonucleotides. For example, the supernatant can be isolated following incubation and the level of a cytokine such as IFNα, IL-6, or IL-12 p40 (or any other suitable cytokine known to be induced as a result of TLR7 or TLR8 signalling) can be determined using, e.g., sandwich ELISA.

TLR7 and TLR8 stimulation can be assessed using any of a number of possible readout systems, most based upon a TLR/IL-1R signal transduction pathway, involving, e.g., MyD88, TRAF, IRAK4, p38, and/or ERK (Hcker H et al. (1999) EMBO J. 18:6973-82). These pathways activate kinases including KB kinase complex and c-Jun N-terminal kinases. TLR7 and TLR8 activation can be assessed by examining any aspect of TLR signaling. For example, activation of TLR signaling triggers alterations in protein-protein associations (e.g., IRAK with MyD88 and/or TRAF6), in protein activity (e.g., kinase activity of proteins such as TAK-1), in intracellular localization of proteins (such as movement of NK-kB into the nucleus), and in gene expression (e.g., in expression of NK-kB sensitive genes), and cytokine production (e.g., production and secretion of IFNα, IL-6 and/or IL-12 p40). Any such alteration can be detected and used to detect TLR7 or TLR8 activation. In a particularly preferred embodiment, TLR7 stimulation is detected by collecting supernatants after 18-20 hr of culture and measuring levels of IFNα, IL-6 and/or IL-12 p40 by sandwich ELISA. In another preferred embodiment, TLR8 stimulation is detected by collecting supernatants after 18-20 hr of culture and measuring levels of IL-6, TNF-α and/or IL-12 p40 by sandwich ELISA.

In another embodiment, cells are used that contain a reporter construct that causes the expression of a detectable gene product upon TLR7 or TLR8 stimulation and consequent activation of the signal transduction pathway. Reporter genes and reporter gene constructs particularly useful for the assays include, e.g., a reporter gene operatively linked to a promoter sensitive to NF-kB. Examples of such promoters include, without limitation, those for IL-11, IL-6, IL-8, IL-12 p40, IP-10, CD80, CD86, and TNF-α. The reporter gene operatively linked to the TLR-sensitive promoter can include, without limitation, an enzyme (e.g., luciferase, alkaline phosphatase, β-galactosidase, chloramphenicol acetyltransferase (CAT), etc.), a bioluminescence marker (e.g., green-fluorescent protein (GFP, e.g., U.S. Pat. No. 5,491,084), blue fluorescent protein (BFP, e.g., U.S. Pat. No. 6,486,382), etc.), a surface-expressed molecule (e.g., CD25, CD80, CD86), and a secreted molecule (e.g., IL-1, IL-6, IL-8, IL-12 p40, TNF-α.). See, e.g., Hcker H et al. (1999) EMBO J. 18:6973-82; Murphy T L et al. (1995) Mol Cell Biol 15:5258-67, the disclosures of which are herein incorporated by reference. TLR signaling reporter plasmids are commercially available (InvivoGen, San Diego, Calif.).

In assays relying on enzyme activity readout, substrate can be supplied as part of the assay, and detection can involve measurement of chemoluminescence, fluorescence, color development, incorporation of radioactive label, drug resistance, optical density, or other marker of enzyme activity. For assays relying on surface expression of a molecule, detection can be accomplished using flow cytometry (FACS) analysis or functional assays. Secreted molecules can be assayed using enzyme-linked immunosorbent assay (ELISA) or bioassays. Many of these and other suitable readout systems are well known in the art and are commercially available. Preferably, the reporter system, whichever used, is quantifiable.

Oligonucleotides are said to be stimulating if they induce any detectable alteration in the marker used to assess TLR7- or TLR8-mediated activity. For example, the oligonucleotide can cause an alteration in the marker expression, activity, phosphorylation, secretion, etc., of 5%, 10%, 20%, 30%, 40%, 50%, 60%, 70%, 80%, 90%, 100%, 150%, 200%, 300%, 400%, 500%, 1000%, or greater.

The herein-described oligonucleotides can be used in such assays to identify novel modulators of TLR7 and/or TLR7-expressing cells. Generally, the screening methods involve assaying for compounds which inhibit or enhance signaling through TLR7. The methods employ TLR7, a suitable reference ligand for the TLR (e.g., one of the herein-described oligonucleotides such as polyUs-21), and a candidate modulating compound. Typically, the TLR7 is contacted with the reference oligonucleotide and a TLR-mediated reference signal is measured. The selected TLR is also contacted with the candidate compound and a TLR-mediated test signal is measured. The test signal and the reference signal are then compared. A favorable candidate compound may subsequently be used as a reference compound in the assay. Such methods are adaptable to automated, high throughput screening of candidate sequences and oligonucleotide modifications. Examples of such high throughput screening methods are described in U.S. Pat. Nos. 6,103,479; 6,051,380; 6,051,373; 5,998,152; 5,876,946; 5,708,158; 5,443,791; 5,429,921; and 5,143,854. In an identical manner, the TLR-8 activating oligonucleotides of this invention can be used to identify modulators of TLR8 and/or TLR8-expressing cells.

### Compositions

The invention provides compositions comprising one or more of the oligonucleotides of this invention and an acceptable carrier. Preferably, a composition of this invention comprises an effective amount of a) a single-stranded oligonucleotide consisting of between i) 10 and 50 nucleotides and comprising a sequence selected from: UUU-(X)n-UUU, or UU-X-UU-X-UU, or Y(U)pY, wherein: each U is independently selected from a uracil-containing nucleotide; each X is independently selected from any nucleotide; n is an integer from 1 to 4; and p is an integer greater than 4; or ii) 11 and 50 nucleotides and comprising a sequence selected from: GGG-(X)n-GGG, GG-X-GG-X-GG, or Z(G)pZ, wherein: each G is independently selected from a guanine-containing nucleotide; each X is independently selected from any nucleotide; each Z is independently selected from any non-guanine nucleotide; n is an integer from 1 to 4; and p is an integer greater than 4; and b) a pharmaceutically-acceptable carrier (a “pharmaceutical composition”).

Pharmaceutically acceptable solutions typically contain pharmaceutically acceptable concentrations of salt, buffering agents, preservatives, compatible carriers, adjuvants, and optionally other therapeutic ingredients. Pharmaceutically acceptable carriers, adjuvants and vehicles that may be used in the pharmaceutical compositions useful in this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in therapy, an effective amount of the compound can be administered to a subject by any mode allowing the compound to be taken up by the appropriate target cells, e.g., pDCs, monocytes, mDCs, Treg cells. “Administering” the pharmaceutical composition of the present invention can be accomplished by any means known to the skilled artisan. The compositions of the present invention may be administered orally, parenterally, by inhalation spray, topically, transdermally, rectally, nasally, buccally, sublingually, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. An injection can be in a bolus or a continuous infusion. Various methods of preparing and administering therapeutic agents are well known in the art and are taught, e.g., in Remington's Pharmaceutical Sciences” 15th Edition, the entire disclosure of which is herein incorporated by reference.

The pharmaceutical compositions are preferably prepared and administered in dose units. Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

Liquid dose units are vials or ampoules for injection or other parenteral administration. Solid dose units are tablets, capsules, powders, and suppositories. For treatment of a patient, depending on activity of the compound, manner of administration, purpose of the administration (i.e., prophylactic or therapeutic), nature and severity of the disorder, age and body weight of the patient, different doses may be necessary. The administration of a given dose can be carried out both by single administration in the form of an individual dose unit or else several smaller dose units. Repeated and multiple administration of doses at specific intervals of days, weeks, or months apart are also contemplated by the invention. The concentration of compounds included in compositions used in the methods of the invention can range from about 1 nM to about 100 μM. Effective doses are believed to range from about 10 picomole/kg to about 100 micromole/kg.

Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein. Methods of formulating such slow or controlled release compositions of pharmaceutically active ingredients, such as those herein and other compounds known in the art, are known in the art and described in several issued U.S. Patents, some of which include, but are not limited to, U.S. Pat. Nos. 4,369,172; and 4,842,866, and references cited therein. Coatings can be used for delivery of compounds to the intestine (see, e.g., U.S. Pat. Nos. 6,638,534, 5,217,720, and 6,569,457, 6,461,631, 6,528,080, 6,800,663, and references cited therein).

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added. Surfactants such as sodium lauryl sulfate may be useful to enhance dissolution and absorption.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be administered in the form of suppositories for rectal or vaginal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition will be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Aerosol formulations that may be utilized in the methods of this invention also include those described in U.S. Pat. No. 6,811,767, the disclosure of which is herein incorporated by reference.

The compositions can be administered per se (neat) or in the form of a pharmaceutically acceptable salt. When used in medicine the salts should be pharmaceutically acceptable, but non-pharmaceutically acceptable salts can conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Other delivery systems can include time-release, delayed release or sustained release delivery systems (collectively referred to herein as “implantable drug release devices”). Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Thus, according to another embodiment, the invention provides a method of impregnating or filling an implantable drug release device comprising the step of contacting said drug release device with a TLR agonist oligonucleotide or a composition comprising a TLR agonist oligonucleotide of this invention.

According to another embodiment, the invention provides an implantable drug release device impregnated with or containing a TLR agonist oligonucleotide or a composition comprising a TLR agonist oligonucleotide of this invention, such that said TLR agonist is released from said device and is therapeutically active.

The present oligonucleotides can also be administered (or used in vitro) along with other compounds designed to enhance their ability to reach or enter cells, to increase their stability in vivo, or for other purposes. In a preferred such embodiment, the oligonucleotides are complexed with a cationic compound such as polyethylenimine (PEI), which binds to and compacts nucleic acids, protecting them from degradation and facilitating their uptake into cells (see, e.g., Boussif et al. (1995) PNAS 92: 7297; Godbey (1999) PNAS 96: 5177). It will be appreciated that, while PEI is preferred, other compaction agents or cationic substances can also be used.

Compaction agents also can be used alone, or in combination with, a biological or chemical/physical vector. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

In other embodiments, the oligonucleotides are complexed with liposomes. Liposomes are useful, inter alia, in that they can be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art.

Liposomes fall into two broad classes. Cationic liposomes are positively charged liposomes which interact with the negatively charged ssRNA molecules to form a stable complex. The positively charged ssRNA/liposome complex binds to the negatively charged cell surface and is internalized in an endosome. Due to the acidic pH within the endosome, the liposomes are ruptured, releasing their contents into the cell cytoplasm (Wang et at., Biochem. Biophys. Res. Commun., 1987, 147, 980-985).

Liposomes which are pH-sensitive or negatively-charged, entrap ssRNA rather than complex with it. Since both the ssRNA and the lipid are similarly charged, repulsion rather than complex formation occurs. The ssRNA is thus entrapped in the aqueous interior of these liposomes. pH-sensitive liposomes have been used, for example, to deliver ssRNA encoding the thymidine kinase gene to cell monolayers in culture (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Liposomes that include nucleic acids have been described, for example, in Thierry et al., WO 96/40062 (methods for encapsulating high molecular weight nucleic acids in liposomes); Tagawa et al., U.S. Pat. No. 5,264,221 (protein-bonded liposomes containing RNA); Rahman et al., U.S. Pat. No. 5,665,710 (methods of encapsulating oligodeoxynucleotides in liposomes); Love et al., WO 97/04787 (liposomes that include antisense oligonucleotides).

Another type of liposome, transfersomes are highly deformable lipid aggregates which are attractive for drug delivery vehicles. (Cevc et al., 1998, Biochim Biophys Acta. 1368(2): 201-15.) Transfersomes may be described as lipid droplets which are so highly deformable that they can penetrate through pores which are smaller than the droplet. Transfersomes are adaptable to the environment in which they are used, for example, they are shape adaptive, self-repairing, frequently reach their targets without fragmenting, and often self-loading. Transfersomes can be made, for example, by adding surface edge-activators, usually surfactants, to a standard liposomal composition.

Lipid formulations for transfection are commercially available from QIAGEN, for example, as EFFECTENE™. (a non-liposomal lipid with a special DNA condensing enhancer) and SUPERFECT™ (a novel acting dendrimeric technology). Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA), DOTAP and dimethyl dioctadecylammonium bromide (DDAB). Methods for making liposomes are well known in the art and have been described in many publications. Liposomes also have been reviewed by, inter alia, Gregoriadis (1985) Trends Biotechnol 3:235-241.

The compositions of the invention may comprise other agents useful for the treatment or prevention of the relevant condition, e.g., cancer or infection such as viral infection.

In one embodiment, the oligonucleotide of this invention is formulated in a composition together with an antigen. The antigen may be present in the composition as a discrete component or, alternatively, conjugated to the oligonucleotide to form a complex. In a complex, the two agents may be either covalently bonded or conjugated directly to one other or attached via a linker or tether moiety. In a preferred embodiment, the antigen is a viral antigen, a cancer antigen or an allergen. Such composition is used to stimulate an antigen-specific response against a disease or condition characterized by that antigen.

As used herein, the term “viral antigen” includes, but is not limited to, intact, attenuated or killed whole virus, any structural or functional viral protein, or any peptide portion of a viral protein of sufficient length (typically about 8 amino acids or longer) to be antigenic. Sources of a viral antigen include, but are not limited to viruses from the families: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviruses and rotaviruses); Bomaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g., African swine fever virus); and unclassified viruses (e.g., the agent of delta hepatitis (thought to be a defective satellite of hepatitis B virus), Hepatitis C; Norwalk and related viruses, and astroviruses). Alternatively, a viral antigen may be produced recombinantly.

As used herein, the terms “cancer antigen” and “tumor antigen” are used interchangeably and refer to antigens that are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses.

A cancer antigen as used herein is a compound, such as a peptide, protein, or glycoprotein, which is associated with a tumor or cancer cell surface and which is capable of provoking an immune response when expressed on the surface of an antigen-presenting cell in the context of a major histocompatibility complex (MHC) molecule. Cancer antigens can be prepared from cancer cells either by preparing crude extracts of cancer cells, for example, as described in Cohen P A et al. (1994) Cancer Res 54:1055-8, by partially purifying the antigens, by recombinant technology, or by de novo synthesis of known antigens. Cancer antigens include but are not limited to antigens that are recombinantly expressed, an immunogenic portion of, or a whole tumor or cancer or cell thereof. Such antigens can be isolated or prepared recombinantly or by any other means known in the art.

Examples of tumor antigens include MAGE, MART-1/Melan-A, gp100, dipeptidyl peptidase IV (DPPIV), adenosine deaminase-binding protein (ADAbp), cyclophilin b, colorectal associated antigen (CRC)-C017-1A/GA733, carcinoembryonic antigen (CEA) and its immunogenic epitopes CAP-1 and CAP-2, etv6, am11, prostate specific antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A1, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, VEGF, VEGF receptors, A-Raf, B-Raf, C-Raf, Raf-1, HSP70, HSP90, PDGF, TGF-alpha, EGF, EGF receptor, a member of the human EGF-like receptor family such as HER-2/neu, HER-3, HER-4 or a heterodimeric receptor comprised of at least one HER subunit, gastrin releasing peptide receptor antigen, Muc-1, CA125, αvβ3 integrins, β5β1 integrins, αIIβ3-integrins, CTLA-4, CD20, CD22, CD30, CD33, CD52, CD56, CD80, PDGF beta receptor, Src, VE-cadherin, IL-8, hCG, IL-6, IL-6 receptor, IL-15, p21ras, RCAS1, α-fetoprotein, E-cadherin, α-catenin, β-catenin and γ-catenin, p120ctn, gp100.sup.Pme1117, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, Smad family of tumor antigens, imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2, or any additional protein target set forth in http://oncologyknowledgebase.com/oksite/TargetTherapeutics/TTOExhibit2.pdf and http://oncologyknowledgebase.com/oksite/TargetedTherapeutics/TTOExhibit3.pdf, the disclosures of which are herein incorporated by reference. This list is not meant to be limiting.

Allergens that may be used in the compositions (and methods) of this invention are too numerous to list. A few examples of such allergens include, but are not limited to, pollens, insect venoms, animal dander dust, fungal spores and drugs (e.g., penicillin). Examples of natural animal and plant allergens include proteins specific to the following genuses: Canis (Canis familiaris); Dermatophagoides (e.g., Dermatophagoides farinae); Felis (Felis domesticus); Ambrosia (Ambrosia artemiisfolia; Lolium (e.g., Lolium perenne and Lolium multiflorum); Cryptomeria (Cryptomeria japonica); Alternaria (Alternaria alternata); Alder; Alnus (Alnus gultinosa); Betula (Betula verrucosa); Quercus (Quercus alba); Olea (Olea europa); Artemisia (Artemisia vulgaris); Plantago (e.g., Plantago lanceolata); Parietaria (e.g., Parietaria officinalis and Parietaria judaica); Blattella (e.g., Blattella germanica); Apis (e.g., Apis multiflorum); Cupressus (e.g., Cupressus sempervirens, Cupressus arizonica and Cupressus macrocarpa); Juniperus (e.g., Juniperus sabinoides, Juniperus virginiana, Juniperus communis, and Juniperus ashei); Thuya (e.g., Thuya orientalis); Chamaecyparis (e.g., Chamaecyparis obtusa); Periplaneta (e.g., Periplaneta americana); Agropyron (e.g., Agropyron repens); Secale (e.g., Secale cereale); Triticum (e.g., Triticum aestivum); Dactylis (e.g., Dactylis glomerata); Festuca (e.g., Festuca elatior); Poa (e.g., Poa pratensis and Poa compressa); Avena (e.g., Avena sativa); Holcus (e.g., Holcus lanatus); Anthoxanthum (e.g., Anthoxanthum odoratum); Arrhenatherum (e.g., Arrhenatherum elatius); Agrostis (e.g., Agrostis alba); Phleum (e.g., Phleum pratense); Phalaris (e.g., Phalaris arundinacea); Paspalum (e.g., Paspalum notatum); Sorghum (e.g., Sorghum halepensis); and Bromus (e.g., Bromus inermis).

As described supra, the present oligonucleotides can be used to treat or prevent any condition that can be beneficially affected by enhanced pDC activity, or by enhanced activity of any TLR7-expressing cells or TLR8-expressing cells, such as allergy, asthma, autoimmune disease, and for any type of weakened immune system resulting from any of a variety of potential causes. It will be appreciated that, regardless of the condition being treated, any other agent that can be used to treat the relevant condition can be present in a composition of this invention together with a herein described TLR agonist oligonucleotide.

In another embodiment, the oligonucleotide of this invention is formulated in a composition together with another therapeutic agent useful in the treatment of cancer. Such agents include agonists of other TLRs (e.g, TLR3, TLR7, TLR8, TLR9); agonists of the same TLR that the oligonucleotide agonizes, but having a different molecular structure (i.e., a different nucleotide sequence); cytotoxic agents, including but not limited to, radioisotopes, toxic proteins, toxic small molecules, such as drugs, toxins, immunomodulators, hormones, hormone antagonists, enzymes, oligonucleotides, enzyme inhibitors, therapeutic radionuclides, angiogenesis inhibitors, chemotherapeutic drugs, vinca alkaloids, anthracyclines, epidophyllotoxins, taxanes, antimetabolites, alkylating agents, antibiotics, COX-2 inhibitors, SN-38, antimitotics, antiangiogenic and apoptotic agents, particularly doxorubicin, methotrexate, taxol, CPT-11, camptothecans, nitrogen mustards, gemcitabine, alkyl sulfonates, nitrosoureas, triazenes, folic acid analogs, pyrimidine analogs, purine analogs, platinum coordination complexes, Pseudomonas exotoxin, ricin, abrin, 5-fluorouridine, ribonuclease (RNase), DNase I, Staphylococcal enterotoxin-A, pokeweed antiviral protein, gelonin, diphtherin toxin, Pseudomonas exotoxin, Pseudomonas endotoxin and others (see, e.g., Remington's Pharmaceutical Sciences, 19th Ed. (Mack Publishing Co. 1995); Goodman and Gilman's The Pharmacological Basis of Therapeutics (McGraw Hill, 2001); Pastan et al. (1986) Cell 47:641; Goldenberg (1994) Cancer Journal for Clinicians 44:43; U.S. Pat. No. 6,077,499; http://oncologyknowledgebase.com/oksite/TargetedTherapeutics/TTOExhibit4.pdf, and http://oncologyknowledgebase.com/oksite/TargetedTherapeutics/TTOExhibit5.pdf, the entire disclosures of which are herein incorporated by reference); agents that target a tumor antigen or a tumor proliferative protein, such as siRNA targeted against VEGF, VEGF receptors, A-Raf, B-Raf, C-Raf, Raf-1, HSP70, HSP90, PDGF, TGF-alpha, EGF, EGF receptor, a member of the human EGF-like receptor family such as HER-2/neu, HER-3, HER-4 or a heterodimeric receptor comprised of at least one HER subunit, carcinoembryonic antigen, gastrin releasing peptide receptor antigen, Muc-1, CA125, αvβ3 integrins, α5β1 integrins, αIIbβ3-integrins, CTLA-4, CD20, CD22, CD30, CD33, CD52, CD56, CD80, PDGF beta receptor, Src, VE-cadherin, IL-8, hCG, IL-6, IL-6 receptor, IL-15, or an mRNA encoding any additional protein targets set forth in http://oncologyknowledgebase.com/oksite/TargetedTherapeutics/TTOExhibit2.pdf, and http://oncologyknowledgebase.com/oksite/TargetedTherapeutics/TTOExhibit3.pdf, the disclosures of which are herein incorporated by reference; chemotherapy agents including, but not limited to, cisplatin (CDDP), carboplatin, oxaliplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tarnoxifen, raloxifene, estrogen receptor binding agents, taxol, gemcitablen, navelbine, farnesyl-protein tansferase inhibitors, transplatinum, 5-fluorouracil, vincristin, vinblastin and methotrexate, or any analog or derivative variant of the foregoing; therapeutic agents and combinations of therapeutic agents for treatment of specific cancers, such as for breast cancer: doxorubicin, epirubicin, the combination of doxorubicin and cyclophosphamide (AC), the combination of cyclophosphamide, doxorubicin and 5-fluorouracil(CAF), the combination of cyclophosphamide, epirubicin and 5-fluorouracil (CEF), Herceptin™), tamoxifen, the combination of tamoxifen and a cytotoxin, taxanes including docetaxel and Paclitaxel, the combination of a taxane plus doxorubicin and cyclophophamide; for colon cancer: the combination of 5-FU and leucovorin, the combination of 5FU and levamisole, irinotecan (CPT-11) or the combination of irinotecan, 5-FU and leucovorin (IFL) or oxaliplatin; for prostate cancer: a radioisotope (i.e., palladium, strontium-89 and Iridium), leuprolide or other LHR agonists, nonsteroidal antiandrogens (flutamide, nilutamide, and bicalutamide), steroidal antiandrogens (cyproterone acetate), the combination of leuprolide and flutamide, estrogens such as DES, chlorotrianisene, ethinyl estradiol, conjugated estrogens U.S.P., DES-diphosphate, second-line hormonal therapies such as aminoglutethimide, hydrocortisone, flutamide withdrawal, progesterone, and ketoconazole, low-dose prednisone, or other chemotherapy agents or combination of agent reported to produce subjective improvement in symptoms and reduction in PSA level including docetaxcl, paclitaxel, estramustine/docetaxel, estramustine/etoposide, estramustine/vinblastine, and estramustine/Paclitaxel; for melanoma: dacarbazine (DTIC), nitrosoureas such as carmustine (BCNU) and lomustine (CCNU), agents with modest single agent activity including vinca alkaloids, platinum compounds, and taxanes, the Dartmouth regimen (cisplatin, BCNU, and DTIC), interferon alpha (IFN-α), and interleukin-2 (IL-2); for ovarian cancer: Paclitaxel, docetaxel, cisplatin, oxaliplatin, hexamethylmelamine, tamoxifen, ifosfamide, the combination of paclitaxel (Taxol) or docetaxel (Taxotere) and cisplatin or carboplatin, the combination of cyclophosphamide and cisplatin, the combination of cyclophosphamide and carboplatin, the combination of 5-fluorouracil (5FU) and leucovorin, etoposide, liposomal doxorubicin, gerucitabine or topotecan; for lung cancer: cisplatin, vincristine, vinblastine, mitomycin, doxorubicin, and etoposide, alone or in combination, the combination of cyclophosphamide, doxorubicin, vincristine/etoposide, and cisplatin (CAV/EP), the combination of cisplatin and vinorelbine, paclitaxel, docetaxel or gemcitabine, and the combination of carboplatin and paclitaxel.

The oligonucleotide compositions of this invention may also comprise an anti-angiogenic agent. Such agents include, but are not limited to small molecule inhibitor, neutralizing antibodies, antisense strategies, siRNA, RNA aptamers and ribozymes against VEGF-related gene family proteins; variants of VEGF with antagonistic properties (i.e., as described in WO 98/16551, specifically incorporated herein by reference); agents listed in Table D of U.S. Pat. No. 6,524,583, the disclosure of which agents and indications are specifically incorporated herein by reference; agents that inhibit signaling by a receptor tyrosine kinase including but not limited to VEGFR1, VEGFR-2,3 PDGFR-beta, Flt-3, c-Kit, p38 alpha and FGFR-1; agents that inhibit one or more of the various regulators of VEGF expression and production, such as EGFR, HER-2, COX-2, or HIF-1α; thalidomide or its analogue CC-5013; Bevacuzimab (mAb, inhibiting VEGF-A, Genentech); IMC-1121B (mAb, inhibiting VEGFR-2, ImClone Systems); CDP-791 (Pegylated DiFab, VEGFR-2, Celltech); 2C3 (mAb, VEGF-A, Peregrine Pharmaceuticals); PTK-787 (TKI, VEGFR-1, -2, Novartis); AEE788 (TKI, VEGFR-2 and EGFR, Novartis); ZD6474 (TKI, VEGFR-1, -2, -3 EGFR AstraZeneca); AZD2171 (TKI, VEGFR-1, -2, AstraZeneca); SU11248 (TKI, VEGFR-1-2/PDGFR Pfizer); AG13925 (TKI , VEGFR-1, -2, Pfizer); AG013736 (TKI, VEGFR-1-2, Pfizer); CEP-7055 (TKI, VEGFR-1, -2, 3, Cephalon); CP-547,632 (TKI, VEGFR-1, -2, Pfizer); VEGF-trap (Soluble hybrid receptor VEGF-A, PlGF placenta growth factor) Aventis/Regeneron); GW786024 (TKI, VEGFR-1, -2, -3, GlaxoSmithKline); Bay 93-4006 (TKI, VEGFR-1, -2, PDGFR Bayer/Onyx)- and -AMG706 (TKI; VEGFR-1, -2, -3, Amgen).

The oligonucleotide compositions may also include other therapeutic agents such as immunomodulatory agents such as tumor necrosis factor, interferon alpha, beta, and gamma, IL-2, IL-12, IL-15, IL-21, CpG-containing single-stranded DNA, agonists of other TLRs, other cytokines and immunosuppression agents; F42K and other cytokine analogs; or MIP-1, MIP-1 beta, MCP-1, RANTES, and other chemokines; agents that affect the upregulation of cell surface receptors and GAP junctions; cytostatic and differentiation agents; or inhibitors of cell adhesion.

In yet another embodiment, the oligonucleotide compositions may additionally comprise an anti-viral agent. Useful anti-viral agents that can be used in combination with the molecules of the invention include, but are not limited to, protease inhibitors, nucleoside reverse transcriptase inhibitors, non-nucleoside reverse transcriptase inhibitors and nucleoside analogs. Examples of antiviral agents include but are not limited to zidovudine, acyclovir, gangcyclovir, vidarabine, idoxuridine, trifluridine, and ribavirin, as well as foscarnet, amantadine, rimantadine, saquinavir, indinavir, amprenavir, lopinavir, ritonavir, the alpha-interferons; adefovir, clevadine, entecavir, pleconaril.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardley, N.Y., 1970, 537. An effective amount of a compound of this invention can range from about 0.001 mg/kg to about 1000 mg/kg, more preferably 0.01 mg/kg to about 100 mg/kg, more preferably 0.1 mg/kg to about 10 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents.

For pharmaceutical composition that comprise additional therapeutic agents, an effective amount of the additional therapeutic agent is between about 20% and 100% of the dosage normally utilized in a monotherapy regime using just that additional agent. Preferably, an effective amount is between about 70% and 100% of the normal monotherapeutic dose. The normal monotherapuetic dosages of these additional therapeutic agents are well known in the art. See, e.g., Wells et al., eds., Pharmacotherapy Handbook, 2.sup.nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

It is expected that some of the additional therapeutic agents listed above will act synergistically with the compounds of this invention. When this occurs, its will allow the effective dosage of the additional therapeutic agent and/or the compound of this invention to be reduced from that required in a monotherapy. This has the advantage of minimizing toxic side effects of either the additional therapeutic agent of a compound of this invention, synergistic improvements in efficacy, improved ease of administration or use and/or reduced overall expense of compound preparation or formulation.

It will be recognized by those of skill in the art that certain therapeutic agents set forth above fall into two or more of the categories disclosed above. For the purpose of this invention, such therapeutic agents are to be consider members of each of those categories of therapeutics and the characterization of any therapeutic agent as being in a certain specified category does not preclude it from also being considered to be within another specified category.

In yet another embodiment, the invention provides a composition of matter comprising a TLR7 or TLR8 agonist and another agent selected from: a therapeutic agent useful in the treatment of cancer, a therapeutic agent useful in the treatment of infectious disease, a cancer antigen, a viral antigen or an allergen; in separate dosage forms, but associated with one another. The term “associated with one another” as used herein means that the separate dosage forms are packaged together or otherwise attached to one another such that it is readily apparent that the separate dosage forms are intended to be sold and administered as part of the same regimen. The agent and the TLR7 agonist are preferably packaged together in a blister pack or other multi-chamber package, or as connected, separately sealed containers (such as foil pouches or the like) that can be separated by the user (e.g., by tearing on score lines between the two containers).

In still another embodiment, the invention provides a kit comprising in separate vessels, a) a TLR7 agonist or a TLR8 agonist of this invention; and b) another agent selected from: a therapeutic agent useful in the treatment of cancer, a therapeutic agent useful in the treatment of infectious disease, a cancer antigen, a viral antigen or an allergen.

### Methods of Treatment

In numerous embodiments of the present invention, a single stranded, uridine-rich or guanidine-rich oligonucleotide of the invention will be administered in a therapeutically or prophylactically effective amount to a patient or individual in order to achieve a specific outcome. Accordingly, the present invention provides methods of using the herein-described oligonucleotides for immunostimulation useful in the treatment or prevention of disorders where an enhanced immune response is useful and/or required, such as cancer or infectious disease, e.g., viral infection. Such methods comprise the step of administering to a patient a composition comprising an oligonucleotide of this invention. It will be appreciated that the present oligonucleotides can be used to treat or prevent any condition that can be beneficially affected by enhanced pDC activity, enhanced monocyte activity, enhanced mDC activity, enhanced Treg cell activity, or by enhanced activity of any TLR7- or TLR8-expressing cells. Accordingly, the present methods and compositions can be used to treat or prevent conditions such as allergy, asthma, autoimmune disease, and also to generally enhance immune function in patients with a weakened immune system resulting from disease, surgery, or administration of immunosuppressive agents such as chemotherapeutic agents or other drugs or treatments.

In certain embodiments, the method of stimulating an immune response in a subject according to the invention comprises the additional step of detecting immune cell activity in the subject following the administration of a composition comprising an oligonucleotide of this invention. The detection of activity is preferably performed on dendritic cells, monocytes, or Treg cells obtained from the subject after a period of time following administration of the oligonucleotide composition. The cells may be obtained from the peripheral blood, spleen, bone marrow or lymph node of the subject, preferably from peripheral blood or bone marrow. The cells should be obtained after the oligonucleotide in the administered composition has had sufficient time to affect the immune cells in the subject. Typically, this will be between 1 and 48 hours following administration. Peripheral blood and/or marrow is preferably further purified by known techniques. Typically, the technique is a cell sorting technique, such as fluorescence-activated or magnetic-activated cell sorting using an appropriate reagent specific for the type of cell to be assayed.

The activity of the obtained immune cells will be determined by measuring an activity known to be affected in a particular cell by agonism of TLR7 or TLR8. For determining TLR7 activation, the preferred cell to test is a pDC from the subject. An isolated pDC or population of pDCs is assayed by examining the level of expression of a cytokine selected from the group consisting of IFNα, IL-6, and IL-12 p40. For determining TLR8 activation, the preferred cell to test is selected from a mDC, a monocyte or a Treg cell. For assaying mDCs or monocytes, the level of expression of TNFα, IL-6, or IL-12 p40 is measured. For Treg cells, the assay used preferably examines the level of expression of IL-10 or transforming growth factor 13, or the ability of such cells to suppress the proliferation of naïve CD4⁺ T-cells in a co-culture.

Any of a large number of types of cancer can be treated or prevented using the present oligonucleotides. Essentially, any cancer (or other condition) that can be treated, slowed in its progression, or prevented, by an increase in the activity of pDCs, mDCs, monocytes, Treg cells or other TLR7- or TLR8-expressing cell can be treated. Examples of cancer types or proliferative diseases that can be treated include carcinoma, including that of the bladder, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma.

In one embodiment for treating cancer, a sample of TLR7- or TLR8-expressing cells is obtained from the patient prior to the administration of the oligonucleotides, and the ability of one or more of the present oligonucleotides to activate the cells will be assessed on a portion of that sample. Once a suitable, active oligonucleotide has been identified, it can be used to activate the remaining portion or another sample of the patient's TLR7- or TLR8 expressing cells (which are optionally expanded ex vivo prior to activation) ex vivo, in which the oligonucleotide is applied to the cells in vitro and the activated cells then returned to the patient. Alternatively, following the assessment of activation potential, the patient's cell can be activated in vivo, in which the oligonucleotide (in an appropriate pharmaceutical formulation) is directly administered to the patient. In one embodiment, a sample of pDCs or other TLR7- or TLR-8 expressing cells is subsequently (following administration of the oligonucleotide) obtained from the patient to assess their activity in vivo. The activity can be assessed using any of the methods described supra, e.g. cytokine production, TLR signaling induced gene expression, affect on the proliferation of other cells, etc. In such embodiments, a detection that the pDCs or other TLR-expressing cells are active (or have undergone increased proliferation) is an indication that the oligonucleotide is having the desired effect.

When cancer is being treated using the present oligonucleotides, in another embodiment the method of the present invention comprises the additional step of administering to said patient another anti-cancer compound or subjecting the patient to another therapeutic approache. For solid tumor treatment, for example, the administration of a composition of the present invention may be used in combination with classical approaches, such as surgery, radiotherapy, chemotherapy, and the like. The invention therefore provides combined therapies in which the present oligonucleotides are used simultaneously with, before, or after surgery or radiation treatment; or are administered to patients with, before, or after conventional chemotherapeutic, radiotherapeutic or anti-angiogenic agents, or targeted immunotoxins or coaguligands. When the oligonucleotides are administered to a patient with another agent, the two components may be administered either as separately formulated compositions (i.e., as a multiple dosage form), or as a single composition (such as the combination single dosage forms described above containing an oligonucleotide of this invention and another therapeutic agent).

Examples of other anti-cancer compounds that can be co-administered with the present TLR7- and/or TLR8-stimulating oligonucleotides include cytokines. Various cytokines may be employed in such combined approaches, including any of the cytokines set forth above as useful in combination compositions of this invention. Preferred examples of cytokines include IL-1α IL-1β, IL-2, IL-3, IL-4, IL-5, IL-6, IL-7, IL-8, IL-9, IL-10, IL-11, IL-12, IL-13, IL-15, IL-21, TGF-beta, GM-CSF, M-CSF, G-CSF, TNF-alpha, TNF-beta, LAF, TCGF, BCGF, TRF, BAF, BDG, MP, LIF, OSM, TMF, PDGF, IFN-alpha, IFN-beta, IFN-gamma. Particularly preferred are cytokines that stimulate NK cell cytotoxic activity, such as IL-2, IL-12, or IL-15. Cytokines are administered according to standard regimens, consistent with clinical indications such as the condition of the patient and relative toxicity of the cytokine.

In other embodiments, the TLR7- and/or TLR8-stimulating oligonucleotide compositions of the present invention may be administered in combination with a chemotherapeutic or hormonal therapy agent. A variety of hormonal therapy and chemotherapeutic agents may be used in the combined treatment methods disclosed herein, including any of the agents set forth above as useful in combination compositions of this invention. Preferred chemotherapeutic agents contemplated as exemplary include alkylating agents, antimetabolites, cytotoxic antibiotics, vinca alkaloids, for example adriamycin, dactinomycin, mitomycin, caminomycin, daunomycin, doxorubicin, tamoxifen, taxol, taxotere, vincristine, vinblastine, vinorelbine, etoposide (VP-16), 5-fluorouracil (5FU), cytosine arabinoside, cyclophosphamide, thiotepa, methotrexate, camptothecin, actinomycin-D, mitomycin C, cisplatin (CDDP), aminopterin, combretastatin(s) and derivatives and prodrugs thereof. Also contemplated are kinase inhibitors and particularly angiogenesis inhibitors, including for example inhibitors or VEGFR1, VEGFR2, PDGFR, C-KIT and/or one or more raf kinases (e.g. Raf-a, raf-b and/or raf-c). Preferred hormonal agents include for example LHRH agonists such as leuprorelin, goserelin, triptorelin, and buserelin; anti-estrogens such as tamoxifen and toremifene; anti-androgens such as flutamide, nilutamide, cyproterone and bicalutamide; aromatase inhibitors such as anastrozole, exemestane, letrozole and fadrozole; and progestagens such as medroxy, chlormadinone and megestrol.

In another embodiment, the TLR7- and/or TLR8-stimulating oligonucleotide compositions of the present invention may be administered in combination with a therapeutic antibody. In one embodiment, the TLR7- and/or TLR8-stimulating oligonucleotide composition enhances ADCC activity toward a target cell and is administered preferably in combination with the step of administering to said patient an antibody that binds to an antigen on a target cell which is intended to be depleted. Such therapeutic antibodies are often of the IgG1 or IgG3 subtype although other subtypes and modified versions (e.g. modified Fc regions) are envisaged as well. Examples of therapeutic antibodies that can be used advantageously in accordance with the invention are provided in PCT publication no. WO 2005/009465 assigned to Innate Pharma, the disclosure of which is incorporated herein by reference in its entirety.

The present invention also provides a method of treating or preventing an infectious disease in a subject, particularly treating or preventing a viral infection, comprising the step of administering to said patient a composition of this invention. A subject having an infectious disease is a subject that has been exposed to an infectious organism and has acute or chronic detectable levels of the organism in the body. Exposure to the infectious organism generally occurs with the external surface of the subject, e.g., skin or mucosal membranes and/or refers to the penetration of the external surface of the subject by the infectious organism. In addition to viral diseases, the present oligonucleotides can also be used to defend against other types of infectious agents, including bacteria, prions, fungi, and various parasites. See, e.g.; C. G. A Thomas, Medical Microbiology, Bailliere Tindall, Great Britain 1983, the entire disclosure of which is herein incorporated by reference. A subject requiring prevention of a viral infection is a subject who is a candidate for a vaccination against a viral disease. For certain viral diseases, such a subject is a neonate, infant or adolescent. For other viral diseases, the subject is immunocompromised. For other viral diseases, the subject is any member or the population. Viruses treatable using the present oligonucleotides include, but are not limited to, enteroviruses (including, but not limited to, viruses that the family picornaviridae, such as polio virus, coxsackie virus, echo virus), rotaviruses, adenovirus, hepatitis virus. Specific examples of viruses that have been found in humans include but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses) or avian influenza viruses (e.g. H5N1 or related viruses); Bungaviridae (e.g., Hantaan viruses, bunga viruses, phleboviruses and Nairo viruses); Arenaviridae (hemorrhagic fever viruses); Reoviridae (e.g., reoviruses, orbiviurses and rotaviruses); Bimaviridae; Hepadnaviridae (Hepatitis B virus); Parvoviridae (parvoviruses); Papovaviridae (papillomaviruses, polyoma viruses); Adenoviridae (most adenoviruses); Herpesviridae (herpes simplex virus (HSV) 1 and 2, varicella zoster virus, cytomegalovirus (CMV)); Poxyiridae (variola viruses, vaccinia viruses, pox viruses); Iridoviridae (e.g., African swine fever virus); and unclassified viruses (e.g., the etiological agents of spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

As with cancer, the methods of the invention can comprise the addition step of administering to said subject another agent useful for the treatment of infection. Infection medicaments include but are not limited to anti-bacterial agents, anti-viral agents, anti-fungal agents and anti-parasitic agents.

Anti-viral agents are of particular interest, and include compounds that prevent infection of cells by viruses or replication of the virus within the cell. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleoside analogs), maturation of new virus proteins (e.g. protease inhibitors), and budding and release of the virus. Anti-viral agents that may be administered in combination with the oligonucleotides of the present invention are set forth above in the description of oligonucleotide/anti-viral agent combination compositions of the present invention.

Preferred nucleoside analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, and zidovudine (azidothymidine). Another class of anti-viral agents that may be administered with the oligonucleotides of this invention includes cytokines such as interferons, such as alpha and beta-interferon. Also possible is immunoglobulin therapy, including normal immune globulin therapy and hyper-immune globulin therapy. Normal immune globulin therapy utilizes a antibody product which is prepared from the serum of normal blood donors and pooled. This pooled product contains low titers of antibody to a wide range of human viruses, such as hepatitis A, parvovirus, enterovirus (especially in neonates). Hyper-immune globulin therapy utilizes antibodies which are prepared from the serum of individuals who have high titers of an antibody to a particular virus. Examples of hyper-immune globulins include zoster immune globulin (useful for the prevention of varicella in immuno-compromised children and neonates), human rabies immune globulin (useful in the post-exposure prophylaxis of a subject bitten by a rabid animal), hepatitis B immune globulin (useful in the prevention of hepatitis B virus, especially in a subject exposed to the virus), and RSV immune globulin (useful in the treatment of respiratory syncytial virus infections).

When the method of this invention is designed to prevent viral infection, that method typically comprises the additional step of administering to said subject a viral antigen. The choice of viral antigen can be made from the same viral antigens set forth above as useful in combination compositions of the present invention.

When one or more agents are used in combination with the present oligonucleotide-based therapy, there is no requirement for the combined results to be additive of the effects observed when each treatment is conducted separately. Although at least additive effects are generally desirable, any increased anti-cancer or anti-infection effect above one of the single therapies would be of benefit. Also, there is no particular requirement for the combined treatment to exhibit synergistic effects, although this is certainly possible and advantageous.

Effective amounts of the other therapeutic agents useful in the methods of this invention are well known to those skilled in the art. However, it is well within the skilled artisan's purview to determine the other therapeutic agent's optimal effective-amount range. In one embodiment of the invention where another therapeutic agent is administered to an animal, the effective amount of the compound of this invention is less than its effective amount would be where the other therapeutic agent is not administered. In another embodiment, the effective amount of the conventional agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In another embodiment the invention provides any of the above-described oligonucleotides conjugated to a detectable marker. The term “detectable marker” as used herein refers to any molecule that can be quantitatively or qualitatively observed or measured. Examples of detectable markers useful in the conjugated oligonucleotides of this invention are radioisotopes, fluorescent dyes, or a member of a complementary binding pair, such as a member of any one of: and antigen/antibody, lectin/carbohydrate; avidin/biotin; receptor/ligand; or molecularly imprinted polymer/print molecule systems.

The conjugation of such a detectable marker to the oligonucleotide may be achieved by methods well known in the art. Exemplary U.S. patents that describe the preparation of oligonucleotide conjugates include, for example, U.S. Pat. Nos. 4,828,979; 4,948,882; 5,218,105; 5,525,465; 5,541,313; 5,545,730; 5,552,538; 5,578,717; 5,580,731; 5,580,731; 5,591,584; 5,109,124; 5,118,802; 5,138,045; 5,414,077; 5,486,603; 5,512,439; 5,578,718; 5,508,046; 4,587,044; 4,605,735; 4,667,025; 4,752,779; 4,789,737; 4,824,941; 4,835,263; 4,876,335; 4,904,582; 4,958,013; 5,482,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is incorporated by reference herein in its entirety.

The detectable marker conjugated oligonucleotide of this invention can be used to detect binding of the oligonucleotide to the corresponding TLR. Thus according to another embodiment, the invention provides a method of detecting the binding of an oligonucleotide comprising a sequence selected from: a) UUU-(X)n-UUU, or UU-X-UU-X-UU, or Y(U)pY; or b) GGG-(X)n-GGG, GG-X-GG-X-GG, or Z(G)pZ, wherein each U, G, X, n and p is defined as above, to TLR7 or TLR8, said method comprising the steps of contacting a molecule comprising said oligonucleotide conjugated to a detectable marker with a TLR7 or TLR8-containing material; and detecting said detectable marker. A TLR7- or TLR8-containing material may be an isolated TLR7 or TLR8 protein, a fragment of a TLR7 or TLR8 protein comprising a functional oligonucleotide binding domain; or a cell that expresses TLR7 or TLR8. Optionally, the TLR7 agonist oligonucleotides of the invention do not substantially induce signaling through and/or bind to TLR8; optionally the TLR8 agonist oligonucleotides of the invention do not substantially induce signaling through and/or bind to TLR7.

According to another embodiment, the invention provides a method of determining if a test molecule binds to TLR7 or TLR8 comprising the steps of contacting said a conjugate comprising an oligonucleotide comprising a sequence selected from: a) UUU-(X)n-UUU, or UU-X-UU-X-UU, or Y(U)pY; or b) GGG-(X)n-GGG, GG-X-GG-X-GG, or Z(G)pZ, wherein each U, G, X, n and p is defined as above; and a detectable marker; with a TLR7 or TLR8-containing material; quantifying the detectable marker associated with the TLR7 or TLR8-containing material; contacting said conjugate with said TLR7- or TLR-8 containing material in the presence of said test molecule; determining if the presence of said test molecule reduced the amount of detectable marker associated with the TLR7 or TLR8-containing material. A reduction in the amount of detectable marker associated with the TLR7 or TLR8—containing material in the presence of the test molecule indicates that the test molecule binds to TLR7 or TLR8. The test molecule may then be further assayed for its ability to activate TLR7 or TLR8 by any of the assays described previously.

In a related embodiment the invention provides a kit comprising, in separate vessels: a conjugate comprising an oligonucleotide comprising a sequence selected from: UUU-(X)n-UUU, or UU-X-UU-X-UU, or Y(U)pY; or b) GGG-(X)n-GGG, GG-X-GG-X-GG, or Z(G)pZ, wherein each U, G, X, n and p is defined as above; and a detectable marker; and a TLR7 or TLR8-containing material.

### EXAMPLES

Further aspects and advantages of this invention are disclosed in the following experimental section, which should be regarded as illustrative and not limiting the scope of this application.

### Experimental Procedures

Reagents. Poly I:C was from Pharmacia, and polyU was from Sigma (Poole, UK). CpG-containing oligonucleotides 1668 was made at CRUK or purchased from Sigma (Poole, UK). DNA 21-mer oligonucleotides were synthesized at CRUK and RNA oligonucleotides were obtained from Ambion or Thermo Electron. Polytheylenimine (2 kD) was purchased from Sigma-Aldrich. All reagents except polyU were free of endotoxin.

Animals and cells. C57BL/6 were obtained from Charles River UK. TLR7^{-/y} and TLR7^{30 /y} littermate controls mice were bred at the Research Institute for Microbial Disease. Flt3L-DC were generated from bone marrow cell suspensions in RPMI 1640 medium containing 10% fetal calf serum, 2 mM glutamine, 100 units/ml penicillin, 100 μg/ml streptomycin, 50 μM 2-mercaptoethanol and 50 ng/ml murine Flt3L (R&D systems) and were used at day 10 or 11 of cultures.

Activation assays. For stimulation with oligonucleotides, 2×10⁵ Flt3L-DC were seeded in triplicate in 96-well-plates. Oligonucleotides were added and cells were cultured overnight in a final volume of 200 μl. Controls contained medium alone, 0.5 μg/ml CpG 1668, 100 mM loxoribine or 1 μM R848.

For stimulation with oligonucleotides other than CpG 1668, different doses of each test oligonucleotides were diluted in 150 mM NaCl solution and mixed with an equal volume of 150 mM NaCl solution+/−polytheylenimine (PEI; 3 μl/ml PEI were used irrespective of the RNA dose). After 15 min incubation at room temperature, oligonucleotide/PEI complexes were added to cells. Supernatants were collected after 18-20 hr of culture and levels of IFNα, IL-6 and IL-12 p40 were determined by sandwich ELISA.

Human pDC activation assays. R848 and RNA9.2DR complexed to LyoVec were from Invivogen. RNA oligonucleotides were purchased from Sigma Proligo. Human PMBCs were purified from normal human peripheral blood by Ficoll-Hypaque centrifugation. BDCA4⁺ plasmacytoid DC were purified from total PBMC by positive selection using CD304⁺ Microbeads and MiniMacs from MiltenyiBiotec. Oligonucleotides/PEI complexes, prepared as described above, were added to 1-2×10⁵ pDC seeded in duplicate in 96-well-plates. Cells were cultured overnight in a final volume of 200 μl, supernatants were collected after 18-20 hr of culture and levels of IFNα and IL-6 were determined by sandwich ELISA.

### Results

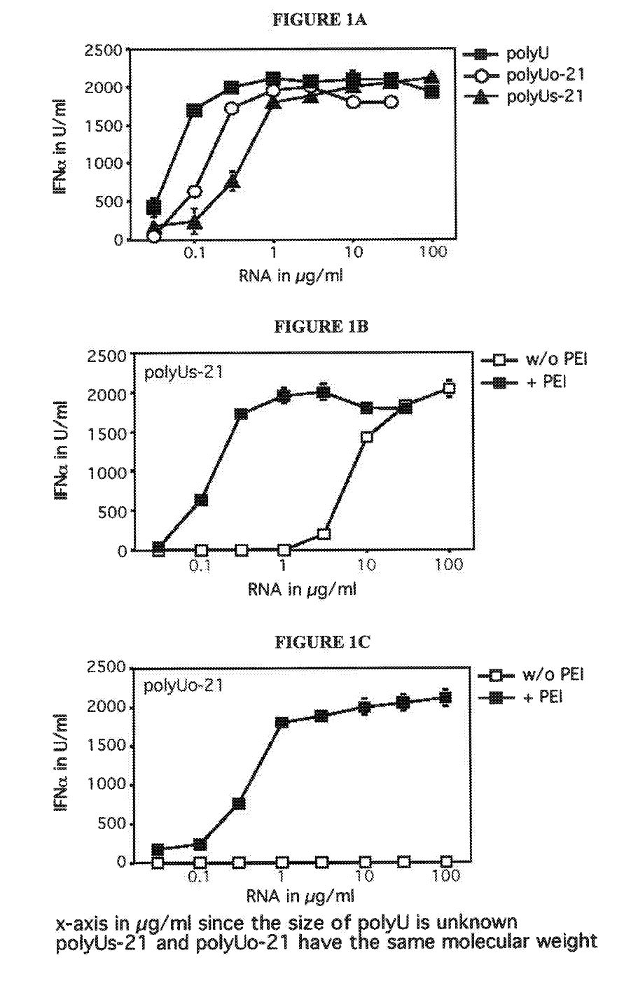

First, we compared IFNα induction by polyU RNA, a previously-studied homopolymer of undefined length, to the induction of IFNα by a 21-mer RNA oligonucleotide with phosphodiester bonds (polyUo-21) as present in natural RNA and in the polyU homopolymer, and by a 21-mer RNA oligonucleotide with phosphorothioate backbone modification (polyUs-21). Both 21-mer polyU oligonucleotides irrespective of the backbone modification induced IFNα by Flt3L-DC in a dose dependent manner (FIG. 1A). Both 21-mer oligonucleotides induced similar IFNα when given to Flt3L culture in form of complexes with PEI and seem to be recognized with equal sensitivity. PEI is a polycation that binds and condenses nucleic acids and thus has the ability to protect RNA from degradation. In addition to this protective function, PEI mediates intracellular uptake of the complexes by a different mechanism than uptake of free RNA.

For all the experiments described here, we used the same concentration of PEI irrespective of the amount of RNA in order to avoid cytotoxicity at higher concentrations of PEI. PEI is not, however, absolutely crucial for uptake and TLR7 recognition of nucleic acid ligands, since polyUs-21 oligonucleotide induced IFNα by Flt3L-DC when given to culture without PEI (FIG. 1B). In contrast, polyUo-21 oligonucleotide failed to induce IFNα under the same conditions (FIG. 1C), which is most likely due to the greater sensitivity of phosphodiester bonds to nuclease digestion. In subsequent experiments, we employed RNA/PEI complexes for stimulation of Flt3L-DC rather than free RNA to avoid differences in stimulatory activity due to differences in the sensitivity to degradation by nucleases.

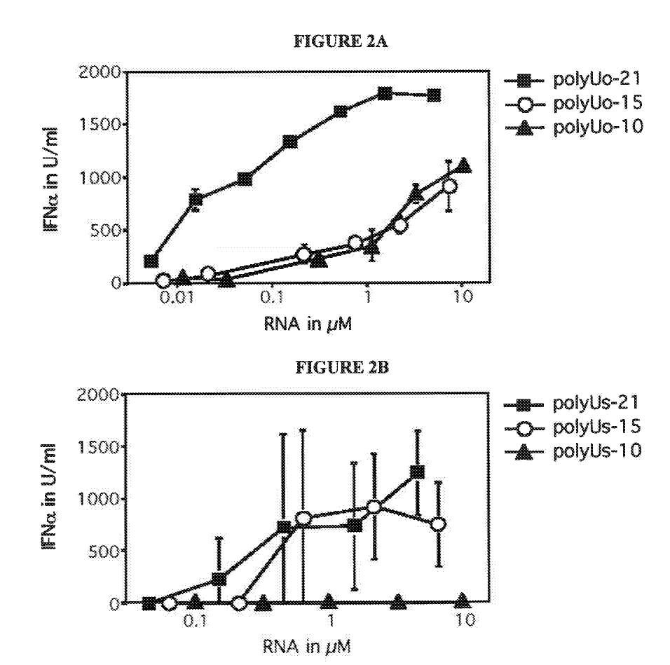

To determine how a reduction of polyU oligonucleotides by 11 and 6 nucleotides affects their stimulatory activity, we compared 10-mer and 15-mer phosphodiester and phosphorothioate polyU RNA oligonucleotides to polyUo-21 and polyUs-21. For phosphodiester polyU RNA, the shorter 15-mer and 10-mer oligonucleotides showed a shift in the dose response and were approximately 20× less potent in inducing IFNα by Flt3L-DC (FIG. 2A). For phosphorothioate polyU RNA, the trend was the same, but the reduction in IFNα induction seen with the 15-mer oligonucleotide was indistinguishable from the response obtained with the 21-mer, whereas the 10-mer didn't induce any measurable levels of IFNα at the tested doses. Thus, we concluded that 10-mer and 15-mer indeed can induce IFNα.

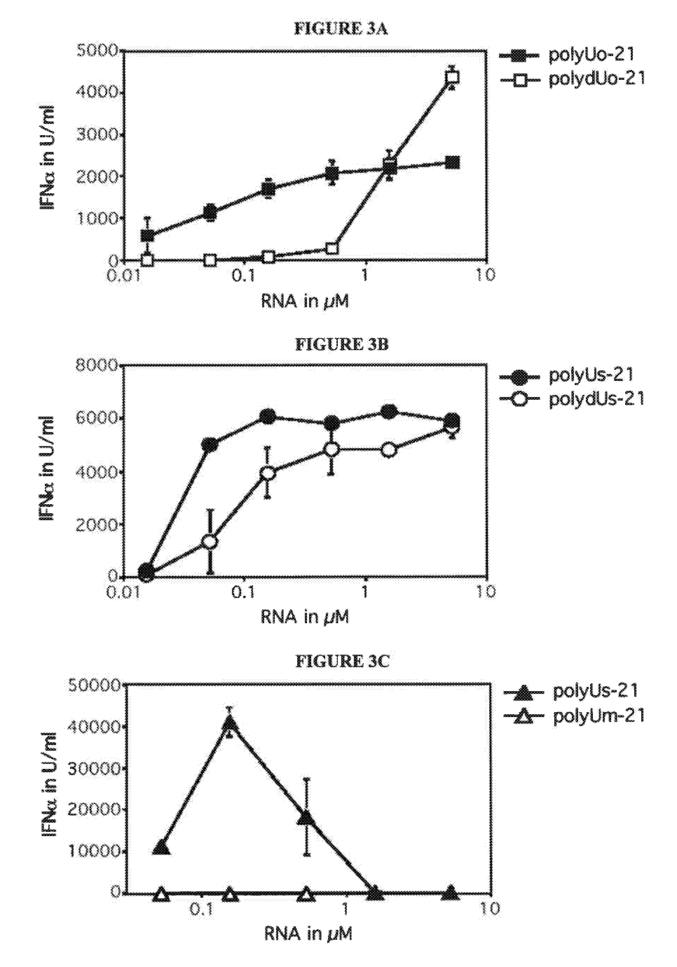

Next we tested whether backbone modifications affect the recognition of ssRNA ligands by TLR7. First we determined whether ssDNA oligonucleotides induce IFNα by Flt3L-DC. When stimulated with a 21-mer polyU phosphodiester DNA oligonucleotide, Flt3L-DC produced IFNα. Interestingly, at lower doses the DNA oligonucleotide (polydUo-21) was less potent than the corresponding RNA oligonucleotide (polyUo-21) in stimulating an IFNα response, whereas at higher doses it was even more potent than polyUo-21 in inducing IFNα (FIG. 3A). When phosphorothioate polyU 21-mer RNA and DNA oligonucleotides were compared (polyUs-21 and polydUs-21, respectively), there was a similar shift in the dose response, but the RNA oligonucleotide induced higher levels of IFNα at all doses tested (FIG. 3B). We concluded that backbone modification at the C2 position of the sugar affects, but does not abrogate, ligand recognition.

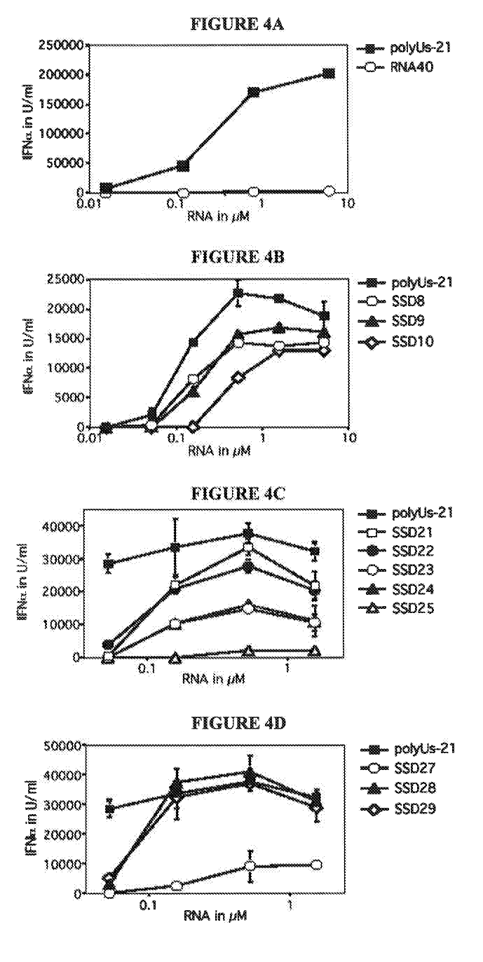

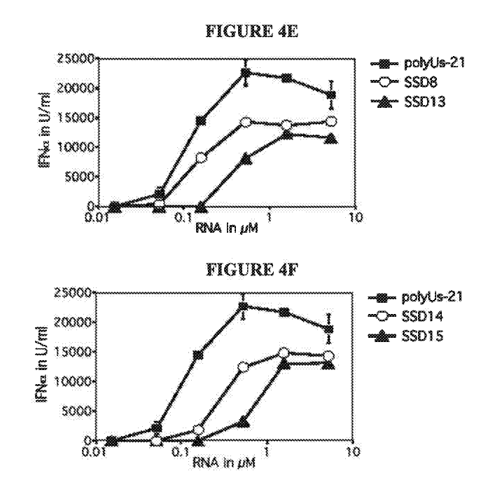

It has been reported that GU-rich motifs in ssRNA are crucial for TLR7 recognition. To address this, we directly compared polyUs-21 to the GU-rich RNA40 oligonucleotide (Heil et al. (2004) Science 303: 1526). In the Flt3L-DC activation assay, the polyUs-21 RNA oligonucleotide was much more potent than RNA40 in inducing IFNα (FIG. 4A). This supports the conclusion that TLR7 exclusively recognizes uridine moieties and ignores all other RNA nucleotides.

To further test our hypothesis that TLR7 recognizes exclusively uridine moieties, we compared 21-mer phosphorothioate RNA oligonucleotides with different compositions of uridine and cytosine moieties. Cytosine moieties were chosen over adenosine to avoid the formation of dsRNA structures by pairing of uridine with adenosine moieties, and were favored over guanosine moieties to avoid GU-rich motifs. For the composition of the different RNA oligonucleotides see Table 1. First we compared 21-mer phosphorothioate oligonucleotides consisting of uridine and cytosine nucleotides and containing either four, three or two triplets of uridines (oligonucleotides SSD8, SSD9 and SSD10, respectively). Differences between oligonucleotides SSD8 and SSD9 were only marginal, while IFNα induction by SSD10 was slightly reduced (FIG. 4B). All three oligonucleotides containing a mixture of uridine and cytosine moieties yielded lower levels of IFNα than the 21-mer oligonucleotide consisting entirely of uridine nucleotides.

In another set of 21-mer phosphorothioate oligonucleotides, we kept the amount of uridine moieties constant at three triplets of uridines, but varied the distance between these three uridine triplets from one cytosine to up to five cytosines (oligonucleotides SSD21-SSD25). The oligonucleotide with the shortest distance between the uridine triplets (SSD21, one cytosine between the triplets) induced the highest levels of IFNα in this group of oligonucleotides, but was, as expected, less efficient in IFNα induction than polyUs-21, which entirely consists of uridine nucleotides (FIG. 4C). The reduction in the levels of IFNα correlated with the increasing distance between the uridine triplets. For oligonucleotide SSD25, for which the distance between the uridine triplets is a stretch of five cytosines, hardly any measurable levels of IFNα were induced (FIG. 4C). To clarify the influence of the distance between distinct uridine moieties on IFNα induction, we tested a second set of 21-mer oligonucleotides that all contained ten uridine nucleotides, but in form of either ten single uridine nucleotides separated by single cytosine nucleotides (SSD27), five double uridine nucleotides separated by single cytosine nucleotides (SSD28) or a stretch of 10 uridine nucleotides flanked by stretches of cytosine nucleotides (SSD29). To our surprise, the oligonucleotides containing a stretch of 10 uridine moieties or 5 doublets of uridine were very potent in inducing IFNα and at higher concentrations yielded levels of IFNα comparable to the polyUs-21 oligonucleotide (FIG. 4D). In contrast to this, the oligonucleotide consisting of alternating uridine and cytosine nucleotides (SSD27) was comparably poor in inducing IFNα in the Flt3L-DC cultures.

To test whether the position of the uridine in the oligonucleotide also affects IFNα induction, we compared 21-mer oligonucleotides with the same number of uridine nucleotides, the same distance between uridine moieties, but with the uridine moieties either positioned at the ends of the oligonucleotides (SSD13 and SSD15) or with no uridine moieties at the ends (SSD8 and SSD14). Two sets of such oligonucleotides were compared and in both cases the oligonucleotide with no uridine moieties at the end were slightly more potent in inducing IFNα and shifted the dose response by nearly half a logarithmic scale (FIGS. 4E and F).

In summary, we concluded from this series of experiments that not only the absolute number of uridine moieties determines the level of IFNα induction, but that the distance between single uridine moieties also influences the induction of IFNα. In addition, the data indicate the uridine moieties at the end of oligonucleotides do not participate to the same extent in IFNα induction as uridine moieties that are located further to the middle of oligonucleotides.

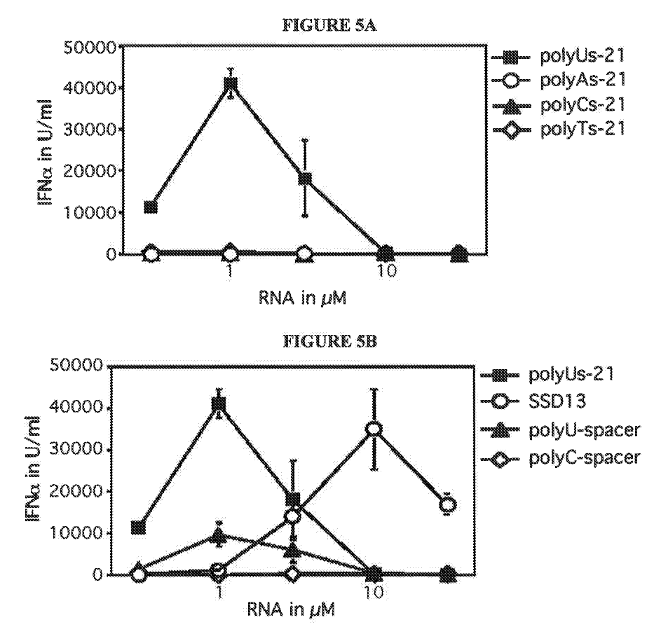

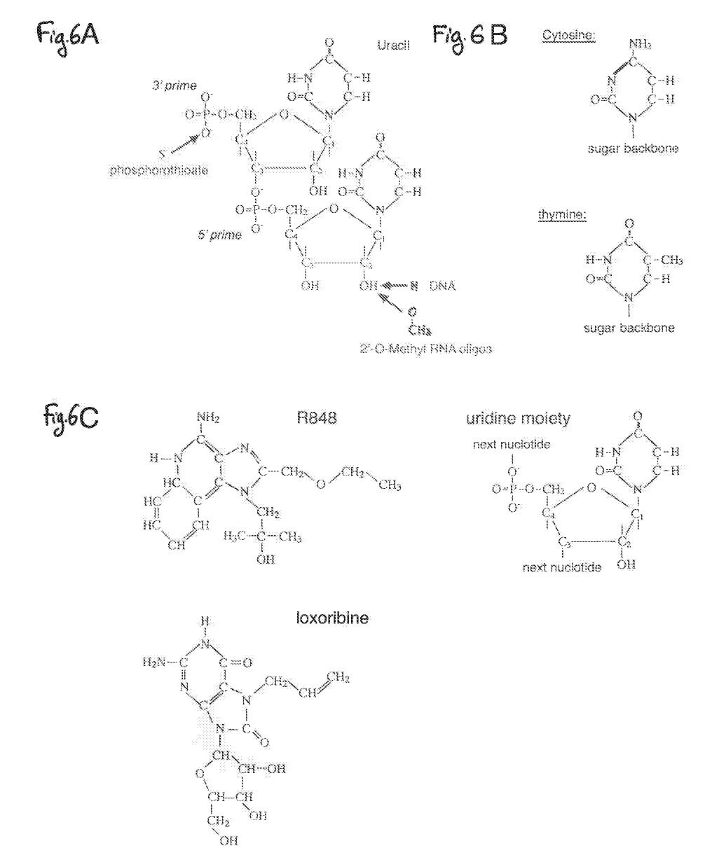

PolyU RNA differs from other RNA homopolymers in that it is unable to form double helical structures at low pH. While other RNA homopolymers can form bonds between two single strands at low pH that are not based on the classical Watson-Crick base pairing of nucleic acids, polyU RNA is unable to do so because of its molecular make-up. Therefore, one possible explanation for the fact that only polyU is recognized by TLR7 is its ability to persist as single-stranded nucleic acid at low pH such as found in the endosomal compartment, where TLR7 recognition takes place. To test this hypothesis in our activation assay with Flt3L-BMDC, we tested the ability of a synthetic 21-mer phosphorothioate polyT RNA oligonucleotide (polyTs-21) to induce IFNα by Flt3L-BMDC. Thymidine differ from uridine nucleotides only by an additional methyl group at the C5 position (FIGS. 6a and B) and, therefore, homopolymeric polyT RNA is, like polyU, unable to form double stranded structures at low pH. Thymidine-containing RNA has never been tested in a TLR7 activation assay, since thymidine nucleotides only from part of DNA and are naturally not present in RNA molecules. Despite the high similarity in structure, polyTs-21 was unable to induce measurable levels of IFNα at any of the tested concentrations (FIG. 5A). Like the polyTs-21 oligonucleotide, the phosphorothioate RNA oligonucleotides polyAs-21 and polyCs-21 also failed to induce IFNα, but this was expected since in previous experiments we had shown that neither polyA nor polyC phosphodiester RNA of undefined length triggered IFNα production.

In a further attempt to test whether the single-stranded nature of polyU RNA is more important for TLR7 activation than the actual polyU moieties, we made use of ribospacer “nucleotides,” which only consist of the sugar/phosphate backbone, but lack a base. We designed oligonucleotides consisting of a mixture of uridine and ribospacer (polyUspacer) or cytidine and ribospacer nucleotides (polyCspacer). Like uridine moieties, ribospacer nucleotides are unable to form bonds between two single RNA strands at low pH that could lead to the formation of double helical structures. We wanted to know whether the polyUspacer would initiate levels of IFNα similar to a polyUs-21, or similar to a oligonucleotide consisting of uridine moieties and other nucleotides that are not recognized by TLR7 (SSD13). We also tested whether the polyCspacer would induce similar levels of IFNα to the oligonucleotide containing uridine and cytosine moieties (SSD13), or whether it would not induce IFNα at all.

When both ribospacer containing oligonucleotides were compared to polyUs-21 and SSD13 oligonucleotides, polyCspacer failed to induce any measurable IFNα at any of the concentrations tested, and polyUspacer induced levels well below those obtained by polyUs-21 or SSD13 oligonucleotide stimulation (FIG. 5B). Taken together, the failure of thymidine and ribospacer “nucleotides” to replace uridine moieties regarding TLR7 stimulation indicates that its not just the single stranded nature of polyU RNA that is preserved at low pH that leads to TLR7 recognition and stimulation, but that it rather the molecular structure of uracil that forms part of the recognition motif for TLR7.

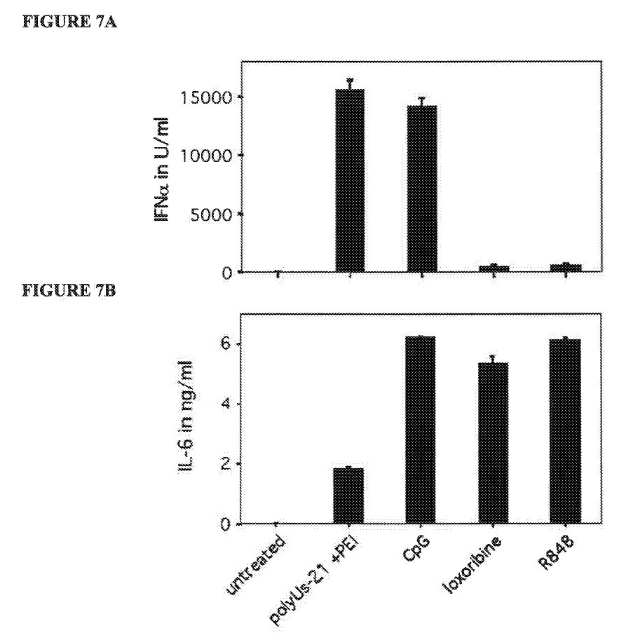

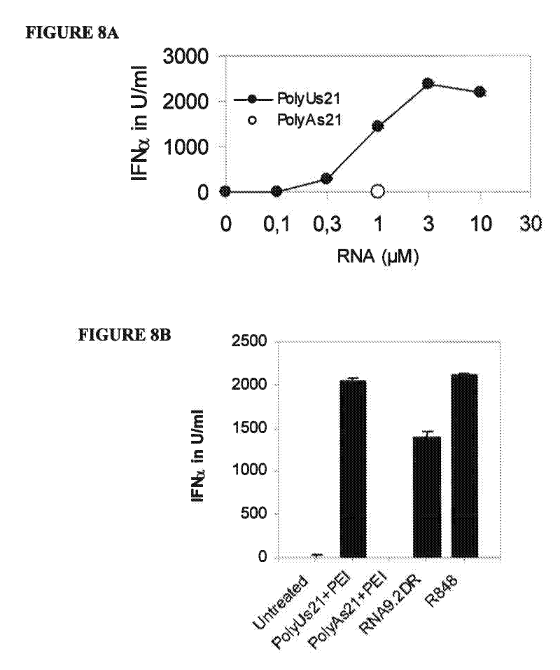

Before viral ssRNA was identified as the natural ligand for TLR7, it had been shown that low molecular weight immune response modifiers such as imidazoquinolines and nucleoside analogues stimulate the innate immune system via a TLR7-dependent pathway. To compare the activity of such small anti-viral compounds with synthetic uridine-rich RNA ligands, Flt3L-DC were cultured in the presence of polyUs-21/PEI complexes, the imidazoquinolin R848 or the substituted guanosine nucleoside loxoribine. As control, cells were treated with the DNA oligonucleotide CpG 1668, which stimulates TLR9. All TLR ligands were used at concentrations that had yielded maximum cytokine induction in previous experiments (data not shown). Surprisingly, the nucleic acid ligands polyUs-21 and CpG were approximately 30 times more potent in inducing IFNα by Flt3L-DC than the low molecular weight anti-viral compounds R848 and loxoribine (FIG. 7A). In contrast to this, the imidazoquinoline and the nucleoside analogue were better inducers of IL-6 than polyUs-21 (FIG. 7B), although the difference in cytokine induction between the RNA ligand and small anti-viral compounds was more dramatic for the induction of IFNα. These results indicate that different TLR7 ligands can have preferences for the inductions of particular cytokines. One possible explanation for this phenomenon is the recruitment of co-receptors to the TLR/ligand complex, which might be more important for stimulation of particular signalling pathways leading to the induction of cytokines such as IFNα. Another possible explanation could be that triggering of the different signalling pathways downstream of TLR7 is influenced by the affinity of the ligand. Although the explanation for this difference in cytokine induction is unclear, it could lead to considerable differences in the type and the strength of the immune response that is induced upon in vivo treatment and therefore could influence the therapeutic outcome.

Short uridine-based homopolymeric oligonucleotides were also assessed for their ability yt oactivate human pDC. Low molecular weight immune response modifiers, such as imidazoquinolines and nucleoside analogues, as well as GU-rich ssRNA have previously been reported to activate human plasmacytoid dendritic cells. To assess whether U-based oligonucleotides can effectively activate human cells, plasmacytoid DC (pDC) were purified from human PBMC and activated with the most potent phosphorothioate-linked RNA oligonucleotide (polyUs-21). As shown in Fig. xA, polyUs21+PEI induced IFN-α production by human pDC in a dose-dependent manner. However, the optimal dose of ssRNA was higher for human pDC than for mouse Flt3L (3 μg/ml and 3 μg/ml respectively). As expected from mouse studies shown above, the phosphorothioate-linked RNA oligonucleotides polyAs-21 failed to induce IFNα (FIG. 9A).

To compare the activity of the different TLR7/8 agonists on human cells, pDC were stimulated with polyUs2/PEI complexes, control polyAs21/PEI complexes, the imidazoquinoline R848, RNA9.2DR oligonucleotides LyoVec complexes. Notably, polyUs21, R848 and RNA9.2DR induced equivalent levels of IFNα production by human pDC (FIG. 9B). Furthermore, while both R848 and RNA9.2DR were good inducers of IL-6 by human pDC, polyUs21 failed to induce IL-6 secretion by pDC (FIG. 9C). As reported above for mouse cells, these results further suggest that different TLR7 agonists can induce distinct cell and cytokine responses.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

TABLE 1

List of RNA oligo with phosphodiester bonds (Uo), phoshorothioate bonds (Us, As, Cs, Gs, Ts),
2′-O-methyl modification (Um) or as DNA oligos (dUs, dUo).

| | | | | | | | | | | | | | | | | | | | | | | | |
|---|---|---|---|---|---|---|---|---|---|---|---|---|---|---|---|---|---|---|---|---|---|---|---|
| | | | | | | | | | | | | | | | | | | | | | | | SEQ |
| RNA Oligos | 1 | 2 | 3 | 4 | 5 | 6 | 7 | 8 | 9 | 10 | 11 | 12 | 13 | 14 | 15 | 16 | 17 | 18 | 19 | 20 | 21 | description | ID NO |

| | | | | | | | | | | | | | | | | | | | | | | | |
|---|---|---|---|---|---|---|---|---|---|---|---|---|---|---|---|---|---|---|---|---|---|---|---|
| polyUo-21 | Uo | Uo | Uo | Uo | Uo | Uo | Uo | Uo | Uo | Uo | Uo | Uo | Uo | Uo | Uo | Uo | Uo | Uo | Uo | Uo | Uo | phosphodiester | 2 |
| | | | | | | | | | | | | | | | | | | | | | | 21-mer | |
| polyUo-15 | Uo | Uo | Uo | Uo | Uo | Uo | Uo | Uo | Uo | Uo | Uo | Uo | Uo | Uo | Uo | | | | | | | phosphodiester | 3 |
| | | | | | | | | | | | | | | | | | | | | | | 15-mer | |
| polyUo-10 | Uo | Uo | Uo | Uo | Uo | Uo | Uo | Uo | Uo | Uo | | | | | | | | | | | | phosphodiester | 4 |
| | | | | | | | | | | | | | | | | | | | | | | 10-mer | |
| polyUs-21 | Us | Us | Us | Us | Us | Us | Us | Us | Us | Us | Us | Us | Us | Us | Us | Us | Us | Us | Us | Us | Us | phosphorothioate | 5 |
| | | | | | | | | | | | | | | | | | | | | | | 21-mer | |
| polyUs-15 | Us | Us | Us | Us | Us | Us | Us | Us | Us | Us | Us | Us | Us | Us | Us | | | | | | | phosphorothioate | 6 |
| | | | | | | | | | | | | | | | | | | | | | | 15-mer | |
| polyUs-10 | Us | Us | Us | Us | Us | Us | Us | Us | Us | Us | | | | | | | | | | | | phosphorothioate | 7 |
| | | | | | | | | | | | | | | | | | | | | | | 10-mer | |
| polydUo-21 | dUo | dUo | dUo | dUo | dUo | dUo | dUo | dUo | dUo | dUo | dUo | dUo | dUo | dUo | dUo | dUo | dUo | dUo | dUo | dUo | dUo | phosphodiester | 8 |
| | | | | | | | | | | | | | | | | | | | | | | DNA 21-mer | |
| polydUs-21 | dUs | dUs | dUs | dUs | dUs | dUs | dUs | dUs | dUs | dUs | dUs | dUs | dUs | dUs | dUs | dUs | dUs | dUs | dUs | dUs | dUs | phosphorothioate | 9 |
| | | | | | | | | | | | | | | | | | | | | | | DNA 21-mer | |
| polyUm-21 | Um | Um | Um | Um | Um | Um | Um | Um | Um | Um | Um | Um | Um | Um | Um | Um | Um | Um | Um | Um | Um | 2′-O-methyl | 10 |
| | | | | | | | | | | | | | | | | | | | | | | modification | |
| RNA40 | Gs | Cs | Cs | Cs | Gs | Us | Cs | Us | Gs | Us | Us | Gs | Us | Gs | Us | Gs | As | Cs | Us | Cs | | Heil et al., | 11 |
| | | | | | | | | | | | | | | | | | | | | | | 2004/20-mer | |
| SSD8 | Cs | Cs | Us | Us | Us | Cs | Cs | Us | Us | Us | Cs | Us | Us | Us | Cs | Cs | Us | Us | Us | Cs | Cs | 4x triple U | 12 |
| SSD9 | Cs | Cs | Cs | Cs | Cs | Cs | Cs | Us | Us | Us | Cs | Us | Us | Us | Cs | Cs | Us | Us | Us | Cs | Cs | 3x triple U | 13 |
| SSD10 | Cs | Cs | Cs | Cs | Cs | Cs | Cs | Us | Us | Us | Cs | Us | Us | Us | Cs | Cs | Cs | Cs | Cs | Cs | Cs | 2x triple U | 14 |
| SSD13 | Us | Us | Us | Cs | Cs | Cs | Us | Us | Us | Cs | Cs | Cs | Us | Us | Us | Cs | Cs | Cs | Us | Us | Us | 4x triple U | 15 |
| SSD14 | Cs | Cs | Us | Us | Cs | Cs | Us | Us | Cs | Cs | Us | Us | Cs | Cs | Us | Us | Cs | Cs | Us | Us | Cs | 5x double U, | 16 |
| | | | | | | | | | | | | | | | | | | | | | | no U at the end | |
| SSD15 | Us | Us | Cs | Cs | Cs | Us | Us | Cs | Cs | Cs | Us | Us | Cs | Cs | Us | Us | Cs | Cs | Cs | Us | Us | 5x double U with | 17 |
| | | | | | | | | | | | | | | | | | | | | | | U at the end | |
| SSD21 | Cs | Cs | Cs | Cs | Cs | Us | Us | Us | Cs | Us | Us | Us | Cs | Us | Us | Us | Cs | Cs | Cs | Cs | Cs | 3x triple U, | 18 |
| | | | | | | | | | | | | | | | | | | | | | | one C apart | |
| SSD22 | Cs | Cs | Cs | Cs | Us | Us | Us | Cs | Cs | Us | Us | Us | Cs | Cs | Us | Us | Us | Cs | Cs | Cs | Cs | 3x triple U, | 19 |
| | | | | | | | | | | | | | | | | | | | | | | two C apart | |
| SSD23 | Cs | Cs | Cs | Us | Us | Us | Cs | Cs | Cs | Us | Us | Us | Cs | Cs | Cs | Us | Us | Us | Cs | Cs | Cs | 3x triple U, | 20 |
| | | | | | | | | | | | | | | | | | | | | | | three C apart | |
| SSD24 | Cs | Cs | Us | Us | Us | Cs | Cs | Cs | Cs | Us | Us | Us | Cs | Cs | Cs | Cs | Us | Us | Us | Cs | Cs | 3x triple U, | 21 |
| | | | | | | | | | | | | | | | | | | | | | | four C apart | |
| SSD25 | Cs | Us | Us | Us | Cs | Cs | Cs | Cs | Cs | Us | Us | Us | Cs | Cs | Cs | Cs | Cs | Us | Us | Us | Cs | 3x triple U, | 22 |
| | | | | | | | | | | | | | | | | | | | | | | five C apart | |
| SSD27 | Cs | Us | Cs | Us | Cs | Us | Cs | Us | Cs | Us | Cs | Us | Cs | Us | Cs | Us | Cs | Us | Cs | Us | Cs | 10x single U, | 23 |
| | | | | | | | | | | | | | | | | | | | | | | one C apart | |
| SSD28 | Cs | Cs | Cs | Cs | Us | Us | Cs | Us | Us | Cs | Us | Us | Cs | Us | Us | Cs | Us | Us | Cs | Cs | Cs | 5x double U, | 24 |
| | | | | | | | | | | | | | | | | | | | | | | one C apart | |
| SSD29 | Cs | Cs | Cs | Cs | Cs | Cs | Us | Us | Us | Us | Us | Us | Us | Us | Us | Us | Cs | Cs | Cs | Cs | Cs | 10x U in a | 25 |
| | | | | | | | | | | | | | | | | | | | | | | string | |
| polyAs-21 | As | As | As | As | As | As | As | As | As | As | As | As | As | As | As | As | As | As | As | As | As | polyA RNA | 26 |
| | | | | | | | | | | | | | | | | | | | | | | 21-mer | |
| polyCs-21 | Cs | Cs | Cs | Cs | Cs | Cs | Cs | Cs | Cs | Cs | Cs | Cs | Cs | Cs | Cs | Cs | Cs | Cs | Cs | Cs | Cs | polyC RNA | 27 |
| | | | | | | | | | | | | | | | | | | | | | | 21-mer | |
| polyTs-21 | Ts | Ts | Ts | Ts | Ts | Ts | Ts | Ts | Ts | Ts | Ts | Ts | Ts | Ts | Ts | Ts | Ts | Ts | Ts | Ts | Ts | polyT RNA | 28 |
| | | | | | | | | | | | | | | | | | | | | | | 21-mer | |
| polyUspacer | Us | Us | Us | Rsp | Rsp | Rsp | Us | Us | Us | Rsp | Rsp | Rsp | Us | Us | Us | Rsp | Rsp | Rsp | Us | Us | Us | Us moieties plus | 29 |
| | | | | | | | | | | | | | | | | | | | | | | ribospacer | |
| polyCspacer | Rsp | Rsp | Rsp | Cs | Cs | Cs | Rsp | Rsp | Rsp | Cs | Cs | Cs | Rsp | Rsp | Rsp | Cs | Cs | Cs | Rsp | Rsp | Rsp | Cs moieties plus | 30 |
| | | | | | | | | | | | | | | | | | | | | | | ribospacer | |

1. A single-stranded oligonucleotide consisting of between 10 and 50 nucleotides and comprising a sequence selected from UUUᵣ-(X)ₙ-UUUᵣ, or UU-X-UU-X-UU, or Y(U)ₚY, wherein:
each U is independently selected from a uracil-containing nucleotide;
each Y is independently selected from a non-uracil-containing nucleotide each X is independently selected from any nucleotide;
r is an integer from 1 to 3;
n is an integer from 1 to 4; and
p is an integer greater than 4; and
wherein said oligonucleotide comprises at least one non-uracil-containing nucleotide or at least one non-natural linkages; or
a single stranded oligonucleotide consisting of between 11 and 50 nucleotides and comprising a sequence selected from: GGG-(X)n-GGG, GG-X-GG-X-GG, or Z(G)pZ, wherein:
each G is independently selected from a guanine-containing nucleotide;
each X is independently selected from any nucleotide;
each Z is independently selected from any non-guanine nucleotide;
n is an integer from 1 to 4; and
p is an integer greater than 4, and
wherein said oligonucleotide comprises at least one non-guanine-containing or at least one non-natural linkage.

2. The oligonucleotide according to claim 1, wherein said oligonucleotide consists of between 10 and 19 nucleotides.

3. The oligonucleotide according to claim 1, wherein said oligonucleotide consists of between 19 and 50 nucleotides.

4. The oligonucleotide according to claim 3, wherein said oligonucleotide consists of between 15 and 30 nucleotides.

5. The oligonucleotide according to claim 4, wherein said oligonucleotide consists of between 21 and 30 nucleotides.

6. The oligonucleotide according to claim 4, wherein said oligonucleotide consists of between 15 and 21 nucleotides.

7. The oligonucleotide according to claim 6, wherein said oligonucleotide consists of 15 nucleotides or 21 nucleotides.

8. The oligonucleotide according to claim 1, wherein said oligonucleotide comprises the sequence UUUᵣ-(X)ₙ-UUUᵣ, r is 2 and n is 1.

9. The oligonucleotide according to claim 1, wherein the oligonucleotide comprises the sequence (UUU-(X)ₙ)ₘ, wherein:
n is an integer from 1 to 4; and
m is an integer greater than 2.

10. The oligonucleotide according to claim 9, wherein n is 1.

11. The oligonucleotide according to claim 9, wherein m is 3 or 4.

12. The oligonucleotide according to claim 1, wherein said oligonucleotide comprises the sequence Y(U)pY, and p is an integer greater than 9.

13. The oligonucleotide according to claim 1, wherein each U is uridine and said oligonucleotide comprises at least one non-natural linkage.

14. The oligonucleotide according to claim 1, selected from a 21-mer comprising one or more phosphorothioate linkages and consisting entirely of uridines; a 21-mer comprising at least 10 consecutive uridines; and a 21-mer comprising the sequence UUXUUXUUXUUXUU, wherein each U is uridine and each X is independently selected from any nucleotide.

15. The oligonucleotide according to claim 1, wherein said oligonucleotide comprises at least 50% uracil-containing nucleotides.

16. The oligonucleotide according to claim 1, wherein said oligonucleotide comprises less than 50% guanine-containing nucleotides.

17. The oligonucleotide according to claim 1, wherein said oligonucleotide is selected from polyUo-21, polyUo-15, polyUo-10 polyUs-21, polyUs-15, polydUo-21, polydUs-21, SSD8, SSD9, SSD10, SSD13, SSD14, SSD15, SSD21, SSD22, SSD23, SSD24, SSD28 or SSD29.

18. The oligonucleotide according to claim 1, wherein said oligonucleotide consists of between 11 and 50 nucleotides and comprises a sequence selected from: GGG-(X)n-GGG, GG-X-GG-X-GG, or Z(G)pZ, wherein:
each G is independently selected from a guanine-containing nucleotide;
each X is independently selected from any nucleotide;
each Z is independently selected from any non-guanine nucleotide;
n is an integer from 1 to 4; and
p is an integer greater than 4,
wherein said oligonucleotide comprises at least one non-guanine-containing nucleotide or at least one non-natural linkage.

19. The oligonucleotide according to claim 18, wherein said oligonucleotide comprises the sequence GGG-(X)n-GGG, and n is 1.

20. The oligonucleotide according to claim 18, wherein said oligonucleotide comprises the sequence (GGG-(X)n)m, wherein:
n is an integer from 1 to 4; and
m is an integer greater than 2.

21. The oligonucleotide according to claim 20, wherein n is 1.

22. The oligonucleotide according to claim 20, wherein m is 3 or 4.

23. The oligonucleotide according to claim 18, wherein said oligonucleotide comprises the sequence Z(G)pZ, and p is an integer greater than 9.

24. The oligonucleotide according to claim 18, wherein each G is guanosine.

25. The oligonucleotide according to claim 18 further comprising a sequence
selected from: UUU-(X)n-UUU, or UU-X-UU-X-UU, or Y(U)pY, wherein:
each U is independently selected from a uracil-containing nucleotide;
each Y is independently selected from any non-uracil containing nucleotide;
each n is independently selected; and
each p is independently selected.

26. The oligonucleotide according to claim 18 further comprising at least one CpG dinucleotide.

27. A pharmaceutical composition comprising:
a. an effective amount of a single-stranded oligonucleotide according to claim 1; and
b. a pharmaceutically-acceptable carrier.

28. (canceled)

29. The pharmaceutical composition according to claim 27, wherein said oligonucleotide is complexed with a compaction agent or a liposome.

30. The pharmaceutical composition according to claim 29, wherein the compaction agent is polyethylenimine.

31. The pharmaceutical composition according to claim 27, further comprising an antigen.

32. The pharmaceutical composition according to claim 31, wherein said antigen is selected from a viral antigen, a cancer antigen or an allergen.

33. The pharmaceutical composition according to claim 27, further comprising a second therapeutic agent.

34. The pharmaceutical composition according to claim 33, wherein said second therapeutic agent is selected from a chemotherapy agent, a radiotherapy agent, a cytotoxin, an anti-angiogenic agent, a monoclonal antibody directed against a cancer antigen, an immunomodulatory agent, a cytokine, an agent that affects the upregulation of cell surface receptors or GAP junctions; a cytostatic or differentiation agent; or a cell adhesion inhibitor, or an antiviral agent.

35. A method of stimulating TLR7 activity in a cell that expresses TLR7, said method comprising the step of contacting the cell with an oligonucleotide according to claim 1.

36. The method according to claim 35, wherein said cell is a plasmacytoid dendritic cell.

37. A method of stimulating TLR8 activity in a cell that expresses TLR8, said method comprising the step of contacting the cell with an oligonucleotide according to claim 18.

38. The method according to claim 37, wherein said cell is selected from a myeloid dendritic cell, a monocyte, or a CD4⁺ regulatory T cell.

39. A method of stimulating an immune response in a subject comprising the step of administering to said patient a composition according to claim 27.

40. (canceled)

41. The method according to claim 39, wherein said method is used to treat or prevent cancer, an infectious disease, allergy, asthma, or an autoimmune disease; or is used to enhance immune function in a patient resulting from disease, surgery, or administration of an immunosuppressive agent.

42. The method according to claim 41, wherein said method is used to treat cancer or to treat or prevent a viral disease.

43. The method according to claim 39, comprising the additional step of detecting immune cell activity of the subject following the administration of the composition.

44. The method according to claim 42, wherein said cancer is selected from carcinoma, including that of the bladder, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma.

45. The method according to claim 44, comprising the additional step of administering to the subject an agent selected from a chemotherapeutic agent, a radiotherapeutic agent, an anti-angiogenic agent, a targeted immunotoxin, a targeted coaguligand, a cytokine, a hormonal therapy agent, or a therapeutic antibody, wherein said agent is administered as a separate dosage form or as part of said composition.

46. The method according to claim 42, whereini said viral disease is caused by a virus in one of the following families: Retroviride, Pircornaviridae, Calciviridae, Togaviridae, Flaviviridae, Coronaviridae, Rhabdoviridae, Filoviridae, Paramyxoviridae, Orthomyxoviridae, Bungaviridae, Arenaviridae, Reoviridae, Birnaviridae, Hepadnaviridae, Parvoviridae, Papovaviridae, Adenoviridae, Herpesviridae, Poxyiridae, Iridoviridae or unclassified viruses.

47. The method according to claim 46, comprising the additional step of administering to the subject an agent selected from a nucleoside analog, a non-nucleotide reverse transcriptase inhibitor, a viral protease inhibitor, an antibody against a viral protein, a viral uncoating agent, or a cytokine, wherein said agent is administered as a separate dosage form or as part of said composition.

48. An implantable drug release device impregnated with or containing a composition comprising according to claim 27, such that said oligonucleotide in said composition is released from said device and is therapeutically active.

49. A method of impregnating or filling an implantable drug release device comprising the step of contacting said drug release device with a composition according to claim 27.

50. A composition of matter comprising:
a. a composition according to claim 27; and
b. a second agent selected from: a therapeutic agent useful in the treatment of cancer, a therapeutic agent useful in the treatment of infectious disease, a cancer antigen, a viral antigen or an allergen;
wherein said composition and said second agent are in separate dosage forms, but associated with one another.

51. A kit comprising in separate vessels:
a. a composition according to claim 27; and
b. a second agent selected from: a therapeutic agent useful in the treatment of cancer, a therapeutic agent useful in the treatment of infectious disease, a cancer antigen, a viral antigen or an allergen.

52. A conjugate comprising:
a. an oligonucleotide according to claim 1; and
b. a detectable marker.

53. A method of detecting the binding of a test oligonucleotide to TLR7 or
a. contacting the conjugate according to claim 52 with a TLR7- or TLR8-containing material, wherein said oligonucleotide portion of said conjugate has the same nucleotide sequence and inter-nucleotide linkages as said test oligonucleotide; and
b. detecting said detectable marker.

54. A method of determining if a test molecule binds to TLR7 or TLR8 comprising the steps of:
a. contacting the conjugate according to claim 52 with a TLR7- or TLR8-containing material in the absence of said test molecule;
b. quantifying the amount of detectable marker bound to said TLR7- or TLR8-containing material;
c. contacting the conjugate according to claim 52 with a TLR7- or TLR8-containing material in the presence of said test molecule; and
d. determining if the presence of said test molecule reduced the amount of detectable marker bound to the TLR7 or TLR8-containing material.

55. The oligonucleotide according to claim 1 further comprising at least one CpG dinucleotide.
